(19) United States
(12) Patent Application Publication
(10) Pub. No.: US 2024/0191185 A1
(43) Pub. Date: Jun. 13, 2024

(54) METHOD FOR PRODUCING PLURIPOTENT STEM CELL POPULATION

(71) Applicant: KANEKA CORPORATION, Osaka-shi, Osaka (JP)

(72) Inventors: Sho KAMBAYASHI, Hyogo (JP); Yoshikazu KAWAI, Hyogo (JP)

(73) Assignee: KANEKA CORPORATION, Osaka-shi, Osaka (JP)

(21) Appl. No.: 18/283,744

(22) PCT Filed: Mar. 25, 2022
(86) PCT No.: PCT/JP2022/014459

§ 371 Date: Sep. 22, 2023

(30) Foreign Application Priority Data
Mar. 25, 2021 (JP) 2021-051004
Mar. 25, 2021 (JP) 2021-051005
Mar. 25, 2021 (JP) 2021-051006

(51) Int. Cl. C12N 5/0735; C12N 5/074

(52) U.S. Cl. CPC C12N 5/0606; C12N 5/0696; C12N 2500/02

(57) ABSTRACT

The pluripotent stem cell culture environment is appropriately controlled to improve production efficiency, and to suppress cell death when passaging pluripotent stem cells from suspension culture. Pluripotent stem cells subjected to suspension culture in a perfusion mode under appropriate medium perfusion conditions are passaged in the subsequent culture step, thereby improving passage efficiency. By strictly controlling the amount of medium perfused in line with the progress of culture of pluripotent stem cells and/or adjusting the amount of carbon dioxide gas supplied in line with the progress of culture of pluripotent stem cells, culture efficiency and productivity can be remarkably improved.

### TECHNICAL FIELD

The present invention relates to a method for producing a cell population in which pluripotent stem cells is to be cultured, and a passage from suspension culture is performed, wherein the amount of medium perfused is strictly controlled and/or the amount of carbon dioxide gas supplied is controlled.

### BACKGROUND ART

Pluripotent stem cells such as ES cells and iPS cells have the ability to grow indefinitely and the ability to differentiate into various somatic cells. The practical application of a therapy in which somatic cells induced to differentiate from pluripotent stem cells is transplanted has the potential to fundamentally revolutionize therapy for incurable diseases and lifestyle-related diseases. For example, technologies have already been developed for inducing the differentiation of pluripotent stem cells into various somatic cells such as nerve cells, myocardial cells, blood cells, and retinal cells in vitro.

Meanwhile, regenerative medicine using pluripotent stem cells still has problems to be solved for practical use, and one of the problems is the productivity of pluripotent stem cells. For example, it is estimated that about 2×10¹¹ cells are required for liver regeneration. Methods for culturing pluripotent stem cells are roughly classified into adherent culture, in which cells are adhered to a flat substrate and cultured, and suspension culture, in which cells are cultured by suspending them in a liquid medium. To culture the said number of cells by adherent culture, a substrate of 10⁶ cm² or more is required, which corresponds to about 20,000 ordinary 10-cm dishes. As such, the number of cells obtained by adherent culture on the substrate surface depends on the culture area, and thus a vast area is required for scale-up, and it is challenging to supply cells in the amount necessary for regenerative medicine. In suspension culture, since cells are cultured while floating in a liquid medium, the number of cells to be obtained depends on the volume of the medium. Therefore, the scale-up of suspension culture is more realistic than adherent culture, and it is suitable for the mass production of cells. For example, Non-Patent Literature 1 discloses a method of suspension culture for pluripotent stem cells while stirring a liquid medium using a spinner flask as a cell culture vessel for suspension culture.

In this connection, as described above, scale-up becomes practical, and mass production of cells becomes possible according to suspension culture, but problems remain regarding production efficiency. For example, there are problems such as cell growth rate, maximum cell density reached, and cell density maintaining rate upon passage. Non-Patent Document 2 discloses a method for improving cell growth by a suspension culture method using a medium perfusion mode. Patent Literature 1 discloses a method of physically fragmenting a cell aggregate formed by suspension culture in a perfusion mode into cells and passaging the cells. Patent Literature 2 discloses a method for culturing PER.C6 (registered trademark) cells by adjusting the amount of perfusion based on a function of cell density. Patent Literature 3 discloses a method for increasing productivity by controlling the aeration volume of carbon dioxide gas during suspension culture of mesenchymal stem cells.

### CITATION LIST

### Non-Patent Literature

Non-Patent Literature 1: Olmer R. et al., Tissue Engineering: Part C, Volume 18 (10): 772-784 (2012)
Non-Patent Literature 2: Kropp C. et al., Stem Cells Translational Medicine, 5: 1289-1301 (2016)

### PATENT LITERATURE

Patent Literature 1: JP 2019-509047 A
Patent Literature 2: JP 2017-225443 A
Patent Literature 3: JP 2016-526894 A

### SUMMARY OF INVENTION

### Technical Problem

Studies have been made to improve the cell growth rate by introducing a perfusion culture method or the like in suspension culture. However, there is no study to improve the efficiency of passage from suspension culture to suspension culture and/or from suspension culture to adherent culture with a focus on the state of cells before passage.

In addition, as described in Patent Literature 2, studies have been made to improve cell productivity by introducing a perfusion culture method to suspension culture.

Further, as described in Patent Literature 3, studies have been made to improve cell productivity by introducing a perfusion culture method to suspension culture, and to improve cell productivity of cells by controlling the volume of carbon dioxide gas supplied. However, in the suspension culture of pluripotent stem cells, which requires delicate control of the culture environment, the control is unsuccessful because the control methods and techniques described in these literatures are insufficient to control the culture environment in some cases. There remain some needs for improvement in culture efficiency through further control of the culture environment.

Therefore, an object of the present invention is to provide a method for producing a pluripotent stem cell population that allows to suppress cell death occurring when passaging pluripotent stem cells from suspension culture and to efficiently culture the cells. Another object of the present invention is to provide a method for producing a pluripotent stem cell population that allows to culture pluripotent stem cells more efficiently by a technology that can respond to environmental changes that may occur during suspension culture of pluripotent stem cells.

### Solution to Problem

As a result of conducting diligent studies to solve the above-described problems, the present inventors have found that it is possible to maintain a highly excellent state of cells immediately before passage and to prevent the pluripotent stem cell death after passage by performing a suspension culture step before the passage using perfusion culture in an appropriate amount of a medium in a method for producing a pluripotent stem cell population by passaging pluripotent stem cells from suspension culture. The present inventors also have found that the culture efficiency can be further improved by using perfusion culture in which the amount of medium perfused is adjusted in line with the progress of culture according to the characteristics of pluripotent stem cells, and by adjusting the amount of carbon dioxide gas supplied in line with the progress of culture. This has led to the completion of the present invention.

The present invention encompasses the following:
(1) A method for producing a pluripotent stem cell population, comprising:
   a step of performing a suspension culture of pluripotent stem cells in a liquid medium in a perfusion mode while adjusting an amount of medium perfused per unit time in a range of 1% to 100% of a culture volume; and
   a step of passaging the pluripotent stem cells subjected to the suspension culture.
(2) The method according to (1), wherein the passage is passage from suspension culture to suspension culture.
(3) The method according to (1) or (2), wherein a cell density one day after the passage is 60% or more of a seeding density for the passage.
(4) The method according to any one of (1) to (3), wherein adjusting the amount of medium perfused per unit time is continuously changing the amount of medium perfused per unit time in accordance with a change in one or more culture variables.
(5) The method according to (4), wherein one of the culture variables is a cell density, and the amount of medium perfused per unit time is increased in line with an increase in the cell density.
(6) The method according to (4) or (5), wherein one of the culture variables is pH, and the amount of medium perfused per unit time is adjusted so as to suppress a decrease in pH.
(7) The method according to any one of (1) to (6), wherein the step of performing suspension culture in a perfusion mode comprises a step of forming a cell aggregate.
(8) The method according to any one of (1) to (7), wherein the step of performing suspension culture in a perfusion mode comprises a step of collecting the cell aggregate.
(9) The method according to any one of (1) to (8), wherein a specific growth rate immediately before the passage is 0.2 to 1.5 day⁻¹.
(10) The method according to any one of (1) to (9), wherein the liquid medium contains at least one selected from the group consisting of L-ascorbic acid, insulin, transferrin, selenium, and sodium hydrogen carbonate.
(11) The method according to any one of (1) to (10), wherein the liquid medium contains FGF2 and/or TGF-β1.
(12) The method according to any one of (1) to (11), wherein the liquid medium contains a ROCK inhibitor.
(13) The method according to (12), wherein the ROCK inhibitor is Y-27632.
(14) The method according to any one of (1) to (13), wherein a proportion of cells positive for OCT4 is 90% or more, a proportion of cells positive for SOX2 is 90% or more, and a proportion of cells positive for NANOG is 90% or more in the pluripotent stem cell population.
(15) The method according to any one of (1) to (14), wherein the pluripotent stem cells are ES cells and/or induced pluripotent stem cells.
(2-1) A method for producing a pluripotent stem cell population, comprising:
   a step of performing suspension culture of pluripotent stem cells in a liquid medium in a perfusion mode while continuously changing an amount of medium perfused per unit time in a range of 1% to 100% of a culture volume in accordance with a change in one or more culture variables.
(2-2) The method according to (2-1), wherein perfusion is started after the pluripotent stem cells form an aggregate.
(2-3) The method according to (2-1) or (2-2), wherein one of the culture variables is a cell density increasing rate with respect to a cell density when the amount of perfusion is started to be changed.
(2-4) The method according to any one of (2-1) to (2-3), wherein one of the culture variables is an aggregate volume increasing rate with respect to a cell aggregate volume when the amount of perfusion is started to be changed.
(2-5) The method according to any one of (2-1) to (2-4), wherein one of the culture variables is a lactic acid production rate of the cells, and the lactic acid production rate is in a range of 1.0×10⁻¹⁰ to 2.5×10⁻⁹ mmol/cell/h.
(2-6) The method according to any one of (2-1) to (2-5), wherein a lactic acid concentration is 10 mM or less when the amount of medium perfused is started to be changed.
(2-7) The method according to any one of (2-1) to (2-6), wherein a seeding density for the suspension culture is 0.01×10⁵ to 20×10⁵ cells/mL.
(2-8) The method according to any one of (2-1) to (2-7), wherein a medium composition used in the perfusion is switched during culture.
(2-9) A method for producing a pluripotent stem cell population, comprising a step of performing suspension culture of pluripotent stem cells in a liquid medium in a perfusion mode while adjusting an amount of medium perfused such that a total amount of medium used for medium change for any 6 hours of culture after a cell density reached 8.0×10⁵ cells/mL becomes greater than a total amount of medium used for medium change for 6 hours of culture immediately before the any 6 hours of culture.
(2-10) The method according to any one of (2-1) to (2-9), wherein the liquid medium contains at least one selected from the group consisting of L-ascorbic acid, insulin, transferrin, selenium, and sodium hydrogen carbonate.
(2-11) The method according to any one of (2-1) to (2-10), wherein the liquid medium contains FGF2 and/or TGF-β1.
(2-12) The method according to any one of (2-1) to (2-11), wherein the liquid medium contains a ROCK inhibitor.
(2-13) The method according to (2-12), wherein the ROCK inhibitor is Y-27632.
(2-14) The method according to any one of (2-1) to (2-13), wherein a cell density at the end of culture in the suspension culture is 1.0×10⁶ cells/mL or more.
(2-15) The method according to any one of (2-1) to (2-14), wherein a proportion of cells positive for OCT4 is 90% or more, a proportion of cells positive for SOX2 is 90% or more, and a proportion of cells positive for NANOG is 90% or more in the pluripotent stem cell population.
(2-16) The method according to any one of (2-1) to (2-15), wherein the pluripotent stem cells are ES cells and/or induced pluripotent stem cells.
(3-1) A method for producing a pluripotent stem cell population, comprising performing suspension culture of pluripotent stem cells while changing an amount of carbon dioxide gas supplied to a liquid medium in accordance with a change in one or more indicators from an arbitrary time point.
(3-2) The method according to (3-1), wherein a method for changing the amount of carbon dioxide gas supplied is a method in which a carbon dioxide gas concentration in a gas supplied to a medium is changed, and the carbon dioxide gas concentration in the gas supplied is in a range of 0% to 10%.
(3-3) The method according to (3-2), wherein the carbon dioxide gas concentration in the gas supplied is started to be changed before the pluripotent stem cells form an aggregate.
(3-4) The method according to any one of (3-1) to (3-3), a mode of medium change for the suspension culture is a perfusion mode, and the suspension culture is performed while continuously changing an amount of medium perfused per unit time in a range of 1% to 100% of a culture volume in accordance with a change in one or more culture variables from an arbitrary time point.
(3-5) The method according to (3-4), wherein perfusion is started after the pluripotent stem cells form an aggregate.
(3-6) The method according to any one of (3-4) to (3-5), wherein a medium composition used in the perfusion is switched during culture.
(3-7) The method according to any one of (3-4) to (3-6), wherein a dissolved carbon dioxide gas concentration in a medium used for the perfusion is lower than a dissolved carbon dioxide gas concentration in the medium.
(3-8) The method according to (3-4) to (3-7), wherein one of the culture variables is pH, and the amount of medium perfused per unit time is adjusted so as to suppress a decrease in pH.
(3-9) The method according to any one of (3-4) to (3-8), wherein one of the culture variables is a cell density, and the amount of medium perfused per unit time is increased in line with an increase in the cell density.
(3-10) The method according to any one of (3-1) to (3-9), wherein one of the culture variables is a lactic acid production rate of the cells, and the lactic acid production rate is in a range of 1.0×10⁻¹⁰ to 2.5×10⁻⁹ mmol/cell/h.
(3-11) The method according to any one of (3-1) to (3-10), wherein the liquid medium contains at least one selected from the group consisting of L-ascorbic acid, insulin, transferrin, selenium, and sodium hydrogen carbonate.
(3-12) The method according to any one of (3-1) to (3-11), wherein the liquid medium contains FGF2 and/or TGF-β1.
(3-13) The method according to any one of (3-1) to (3-12), wherein the liquid medium contains a ROCK inhibitor.
(3-14) The method according to any one of (3-1) to (3-13), wherein a proportion of cells positive for OCT4 is 90% or more, a proportion of cells positive for SOX2 is 90% or more, and a proportion of cells positive for NANOG is 90% or more in the pluripotent stem cell population.
(3-15) The method according to any one of (3-1) to (3-14), wherein the pluripotent stem cells are ES cells and/or induced pluripotent stem cells.
[1] A method for producing a pluripotent stem cell population, comprising:
   a suspension culture step of performing a suspension culture of pluripotent stem cells in a liquid medium in a perfusion mode; and
   a passage step of passaging the pluripotent stem cells subjected to the suspension culture, wherein the suspension culture step comprises controlling an amount of medium perfused per unit time in a range of 1% to 100% of a culture volume.
[2] The method according to [1], wherein the amount of medium perfused per unit time is determined based on a value obtained by multiplying the culture volume by a proportion of a length of the unit time to 24 hours.
[3] The method according to [1] or [2], wherein the control in the suspension culture step is based on one or more culture variables.
[4] The method according to [3], wherein the control allows the amount of medium perfused per unit time to be proportional to each of the one or more culture variables.
[5] The method according to [3] or [4], wherein one of the culture variables is a cell density increasing rate, the control includes increasing the amount of medium perfused per unit time based on an increase in the cell density increasing rate, and the cell density increasing rate represents a proportion of a cell density to a cell density of the pluripotent stem cells at the start of the control.
[6] The method according to any one of [3] to [5], wherein one of the culture variables is a pH of a culture solution in which the pluripotent stem cells are present, and the control includes changing the amount of medium perfused per unit time to suppress a decrease in the pH.
[7] The method according to [6], wherein the decrease in the pH is suppressed by increasing the amount of medium perfused per unit time based on the decrease in the pH.
[8] The method according to claim [6] or [7], wherein the pH during the suspension culture step is maintained at 6.5 or more.
[9] The method according to any one of [3] to [5], wherein one of the culture variables is a lactic acid production rate of the pluripotent stem cells, and the lactic acid production rate is maintained in a range of 1.0×10⁻¹⁰ mmol/cell/h to 2.5×10⁻⁹ mmol/cell/h.
[10] The method according to any one of [1] to [9], wherein a lactic acid concentration in the culture solution in which the pluripotent stem cells are present is 10 mM or less at the start of the control.
[11] The method according to any one of [1] to [10], wherein the pluripotent stem cell population comprises a cell aggregate, and the cell aggregate is collected during the suspension culture step.
[12] The method according to any one of [1] to [11], wherein the pluripotent stem cell population comprises a cell aggregate, and in the suspension culture step, perfusion is started by the perfusion mode after the cell aggregate is formed.
[13] The method according to any one of [1] to [11], wherein the pluripotent stem cell population comprises a cell aggregate, and one of the culture variables is a cell aggregate volume increasing rate, and the cell aggregate volume increasing rate represents a proportion of a cell aggregate volume to a cell aggregate volume at the start of the control.
[14] The method according to any one of [1] to [13], wherein the control includes increasing an amount of medium perfused for any 6 hours of culture after the cell density of the pluripotent stem cells reached 8.0×10⁵ cells/mL compared to an amount of medium perfused for 6 hours of culture immediately therebefore.
[15] The method according to any one of [1] to [14], wherein a culture additive composition of a liquid medium used in perfusion in the perfusion mode is changed during culture.
[16] The method according to any one of [1] to [15], wherein the suspension culture step comprises a altering an amount of carbon dioxide gas supplied to the liquid medium based on one or more indicators.
[17] The method according to [16], wherein the alteration includes changing a carbon dioxide gas concentration in a gas supplied to the liquid medium in a range of 0% to 10%.
[18] The method according to [16], wherein one of the indicators is a pH of the culture solution in which the pluripotent stem cells are present, and the alteration includes changing the carbon dioxide gas concentration in a gas supplied to the liquid medium so as to suppress a decrease in the pH.
[19] The method according to [17] or [18], wherein an average of the carbon dioxide gas concentration for 6 hours is decreased to a range of 0% to 2.5% after one or more days from the start of the alteration.
[20] The method according to any one of [13] to [19], wherein the pluripotent stem cell population comprises a cell aggregate, and the carbon dioxide gas concentration in a gas supplied to the liquid medium is started to be changed before the cell aggregate is formed.
[21] The method according to any one of [1] to [20], wherein a specific growth rate before the passage is 0.2 to 1.5 day⁻¹.
[22] The method according to any one of [1] to [21], wherein a seeding density for the suspension culture step is 0.01×10⁵ cells/mL to 20×10⁵ cells/mL.
[23] The method according to any one of [1] to [22], wherein the cell density of the pluripotent stem cells before the passage is 1.0×10⁶ cells/mL or more.
[24] The method according to any one of [1] to [23], wherein culture is further performed by suspension culture after the passage step.
[25] The method according to any one of [1] to [24], wherein the cell density of the pluripotent stem cells one day after the passage step is 60% or more of the seeding density for the passage step.
[26] The method according to any one of [1] to [25], wherein the liquid medium contains at least one selected from the group consisting of L-ascorbic acid, insulin, transferrin, selenium, and sodium hydrogen carbonate.
[27] The method according to any one of [1] to [26], wherein the liquid medium contains FGF2 and/or TGF-β1.
[28] The method according to any one of [1] to [27], wherein the liquid medium contains a ROCK inhibitor.
[29] The method according to [28], wherein the ROCK inhibitor is Y-27632.
[30] The method according to any one of [1] to [29], wherein a proportion of cells positive for OCT4 is 90% or more, a proportion of cells positive for SOX2 is 90% or more, and a proportion of cells positive for NANOG is 90% or more in the pluripotent stem cell population.
[31] The method according to any one of [1] to [30], wherein the pluripotent stem cells are ES cells and/or induced pluripotent stem cells.

The present specification encompasses the contents described in the specifications and/or drawings of Japanese Patent Application Nos. 2021-051004, 2021-051005, and 2021-051006, on which the priority of the present application is based.

### Advantageous Effects of Invention

According to the present invention, it is possible to efficiently produce a pluripotent stem cell population by preventing the pluripotent stem cell death at passage of pluripotent stem cells from suspension culture and improving the growth efficiency and maximum cell density reached during suspension culture of pluripotent stem cells.

### BRIEF DESCRIPTION OF DRAWINGS

FIG. 1 is a characteristic diagram showing the expression levels of the OCT4 gene, the NANOG gene, the SOX2 gene, and the HK2 gene in the case of suspension culture of pluripotent stem cells by the methods shown in Example A1 and Comparative Examples A1 and A2 of Embodiment A.

FIG. 2 is a characteristic diagram showing the results of flow cytometry analysis of samples treated with anti-OCT4, anti-SOX2, and anti-NANOG antibodies in the case of suspension culture of pluripotent stem cells by the methods shown in Example A1 and Comparative Examples A1 and A2 of Embodiment A.

FIG. 3 is a characteristic diagram showing the analysis results of the cell density immediately before passage for the subsequent suspension culture step in the case of suspension culture of pluripotent stem cells by the methods shown in Example A1 and Comparative Examples A1 and A2 of Embodiment A.

FIG. 4 is a characteristic diagram showing the analysis results of the lactic acid concentration in the culture solution immediately before passage for the subsequent suspension culture step in the case of suspension culture of pluripotent stem cells by the methods shown in Example A1 and Comparative Examples A1 and A2 of Embodiment A.

FIG. 5 is a characteristic diagram showing the analysis results of pH in the culture solution immediately before passage for the subsequent suspension culture step in the case of suspension culture of pluripotent stem cells by the methods shown in Example A1 and Comparative Examples A1 and A2 of Embodiment A.

FIG. 6 is a characteristic diagram showing the analysis results of the specific growth rate for 24 hours immediately before passage for the subsequent suspension culture step in the case of suspension culture of pluripotent stem cells by the methods shown in Example A1 and Comparative Examples A1 and A2 of Embodiment A.

FIG. 7 is a characteristic diagram showing the analysis results of the proportion of the cell density 24 hours after seeding to the seeding concentration (cell density maintaining rate) in the case of passage to suspension culture of pluripotent stem cells suspension-cultured by the methods shown in Example A1 and Comparative Examples A1 and A2 of Embodiment A.

FIG. 8 is a characteristic diagram showing the analysis results of the viable cell rate 24 hours after passage in the case of passage to suspension culture of pluripotent stem cells suspension-cultured by the methods shown in Example A1 and Comparative Examples A1 and A2 of Embodiment A.

FIG. 9 is a characteristic diagram showing the cell density on day 4 of culture in the case of suspension culture of pluripotent stem cells by the methods shown in Example B1 and Comparative Examples B1 and B2 of Embodiment B.

FIG. 10 is a characteristic diagram showing the specific growth rate for day 3 to day 4 of culture in the case of suspension culture of pluripotent stem cells by the methods shown in Example B1 and Comparative Examples B1 and B2 of Embodiment B.

FIG. 11 is a characteristic diagram showing the expression levels of the OCT4 gene, the NANOG gene, and the SOX2 gene on day 2 of culture in the case of suspension culture of pluripotent stem cells by the methods shown in Example B1 and Comparative Examples B1 and B2 of Embodiment B.

FIG. 12 is a characteristic diagram showing the results of flow cytometry analysis of samples treated with anti-OCT4, anti-SOX2, and anti-NANOG antibodies in the case of suspension culture of pluripotent stem cells by the methods shown in Example B1 and Comparative Examples B1 and B2 of Embodiment B.

FIG. 13 is a characteristic diagram showing the cell density on day 6 of culture in the case of suspension culture of pluripotent stem cells by the methods shown in Example B2 and Comparative Examples B3 and B4 of Embodiment B.

FIG. 14 is a characteristic diagram showing the cell density on day 4 of culture in the case of suspension culture of pluripotent stem cells by the methods shown in Comparative Examples C1, C2, and C3 of Embodiment C.

FIG. 15 is a characteristic diagram showing the transition of the concentration of carbon dioxide gas supplied in the case of suspension culture of pluripotent stem cells by the method shown in Example C1 of Embodiment C.

FIG. 16 is a characteristic diagram showing the specific growth rate for day 1 to day 2 of culture in the case of suspension culture of pluripotent stem cells by the methods shown in Example C1 and Comparative Examples C1 and C3 of Embodiment C.

FIG. 17 is a characteristic diagram showing the transition of the concentration of carbon dioxide gas supplied in the case of suspension culture of pluripotent stem cells by the method shown in Example C2 of Embodiment C.

FIG. 18 is a characteristic diagram showing the analysis results of pH as an example of the parameter representing the culture environment in the case of suspension culture by the methods shown in Examples C1 and C2 and Comparative Examples C1, C2, and C3 of Embodiment C.

### DESCRIPTION OF EMBODIMENTS

### 1. Method for Producing Pluripotent Stem Cell Population

### 1-1. Outline

In the method for producing a pluripotent stem cell population according to the present invention, suspension culture of pluripotent stem cells is performed in a liquid medium with medium change, which is generally followed by passage, to produce a pluripotent stem cell population. Optionally, the amount of carbon dioxide gas supplied may be appropriately changed in the liquid medium during the suspension culture. According to the method for producing a pluripotent stem cell population according to the present invention, the cell death occurring in passage of pluripotent stem cells from suspension culture can be prevented, and the growth of pluripotent stem cells can also be promoted, and a pluripotent stem cell population can be produced efficiently.

### 1-2. Definitions of Terms

The following terms used herein are defined.

### <<Cell>>

The “pluripotent stem cell” that is subjected to the invention herein has multipotency (pluripotency) capable of differentiating into all types of cells that constitute the living body, and refers to a cell that can continue to grow indefinitely while maintaining pluripotency in in vitro culture under appropriate conditions. More specifically, pluripotency means the ability to differentiate into germ layers that constitute an individual (three germ layers of ectoderm, mesoderm, and endoderm in vertebrates). Examples of such cells include embryonic stem cells (ES cells), embryonic germ cells (EG cells), germline stem cells (GS cells), and induced pluripotent stem cells (iPS cells).

An “ES cell” is a pluripotent stem cell prepared from an early embryo. “EG cells” refer to pluripotent stem cells prepared from a fetal primordial germ cell (Shamblott M. J. et al., 1998, Proc. Natl. Acad. Sci. USA, 95:13726-13731). A “GS cell” refers to a pluripotent stem cell prepared from a testicular cell (Conrad S., 2008, Nature, 456:344-349). In addition, an “iPS cell” refers to a pluripotent stem cell that can be reprogrammed to an undifferentiated somatic cell by introducing genes encoding a small number of reprogramming factors into a differentiated somatic cell.

A pluripotent stem cell as used herein may be a cell derived from a multicellular organism. A pluripotent stem cell is preferably an animal-derived cell or a mammal-derived cell. mammals include, for example, rodents such as mice, rats, hamsters, and guinea pigs, livestock or pet animals such as dogs, cats, rabbits, bovines, horses, sheep, and goats, and primates such as humans, rhesus monkeys, gorillas, and chimpanzees. For example, human-derived cells may be appropriately used.

The pluripotent stem cell used herein includes a naïve pluripotent stem cell and a primed pluripotent stem cell. A naïve pluripotent stem cell is defined as a cell in a near-pluripotent state found in the pre-implantation inner cell mass. A primed pluripotent stem cell is defined as a cell in a near-pluripotent found in the post-implantation epiblast. A primed pluripotent stem cell is characterized by, compared to naïve pluripotent stem cells, less contribution to ontogenesis, only one transcriptionally active X chromosome, and higher levels of transcriptionally repressive histone modifications. In addition, a primed pluripotent stem cell marker is the OTX2 gene, and naïve pluripotent stem cell markers are the REX1 gene and KLF family gene. Further, the shape of colonies formed by primed pluripotent stem cells is the flat shape, and the shape of colonies formed by naïve pluripotent stem cells is the dome shape. In particular, a primed pluripotent stem cell may be appropriately used as pluripotent stem cells used herein.

The pluripotent stem cell used herein may be a commercially available cell, a distributed cell, or a newly prepared cell. Although not limited, a pluripotent stem cell is preferably an iPS cell or an ES cell when used in each invention described herein.

When an iPS cell used herein is a commercially available iPS cell, a cell line, for example 253G1 strain, 253G4 strain, 201B6 strain, 201B7 strain, 409B2 strain, 454E2 strain, 606A1 strain, 610B1 strain, 648A1 strain, HiPS-RIKEN-1A strain, HiPS-RIKEN-2A strain, HiPS-RIKEN-12A strain, Nips-B2 strain, TkDN4-M strain, TkDA3-1 strain, TkDA3-2 strain, TkDA3-4 strain, TkDA3-5 strain, TkDA3-9 strain, TkDA3-20 strain, hiPSC 38-2 strain, MSC-iPSCT strain, BJ-iPSCT strain, RPChiPS771-2 strain, WTC-11 strain, 1231A3 strain, 1383D2 strain, 1383D6 strain, 1210B2 strain, 1201C1 strain, and 1205B2 strain, may be used, but is not limited thereto.

In addition, when the iPS cell line used herein is a clinical cell line, examples of the clinical cell line that may be used include, but are not limited to, QHJI01s01 strain, QHJI11s04 strain, QHJI14s03 strain, QHJI14s04 strain, Ff-l14s03 strain, Ff-l14s04 strain, and YZWI strain.

When the iPS cell used herein is a newly prepared cell, as a combination of genes of reprogramming factors to be introduced, for example, a combination of the OCT3/4 gene, the KLF4 gene, the SOX2 gene, and the c-Myc gene (Yu J, et al. 2007, Science, 318:1917-20) and a combination of the OCT3/4 gene, the SOX2 gene, the LIN28 gene, and the Nanog gene (Takahashi K, et al. 2007, Cell, 131:861-72) may be used, but is not limited thereto. The form of introducing these genes into cells is not particularly limited, and may be, for example, the introduction of a gene as a nucleic acid, such as gene introduction using a plasmid or introduction of synthetic RNA, or a method using a polymeric compound, such as the introduction as a protein. In addition, an iPS cell produced by a method using microRNA, RNA, a low-molecular-weight compound, or the like may also be used. Further, a clinical grade iPS cell newly prepared by novel techniques may be used.

When an ES cell used herein is a commercially available ES cell, a cell line, for example KhES-1 strain, KhEs-2 strain, KhEs-3 strain, KhEs-4 strain, KhEs-5 strain, SEES1 strain, SEES2 strain, SEES3 strain, SEES-4 strain, SEEs-5 strain, SEEs-6 strain, SEEs-7 strain, HUES8 strain, CyT49 strain, H1 strain, H9 strain, and HS-181 strain, may be used, but is not limited thereto.

### <<Pluripotent Stem Cell Population>>

As used herein, the “pluripotent stem cell population” refers to an assembly of cells composed of one or more cells comprising at least one pluripotent stem cell. A pluripotent stem cell population may consist solely of pluripotent stem cells or comprise other cells. The form of a pluripotent stem cell population is not particularly limited, and includes, for example, a tissue, a tissue fragment, a cell pellet, a cell aggregate, a cell sheet, a solution of suspended cells, a cell suspension, and a frozen product thereof. A pluripotent stem cell population used herein may comprises a plurality of pluripotent stem cell populations of a smaller size. All the small pluripotent stem cell populations contained in the pluripotent stem cell population may not be of the same morphology. In addition, a pluripotent stem cell population used herein may comprise cells in a single cell state. Preferably, the pluripotent stem cell population comprises a cell aggregate.

### <<Cell Aggregate>>

As used herein, the “cell aggregate” refers to an aggregated cell population formed by cell aggregation in suspension culture and is also called a spheroid. A cell aggregate is usually roughly spherical. A cell constituting a cell aggregate is not particularly limited as long as they comprise one or more types of pluripotent stem cells described above. For example, a cell aggregate composed of pluripotent stem cells such as human pluripotent stem cells or human embryonic stem cells comprises cells expressing pluripotent stem cell markers and/or positive for pluripotent stem cell markers.

A pluripotent stem cell marker is a gene marker that is specifically or excessively expressed in pluripotent stem cells. Examples thereof include Alkaline Phosphatase, Nanog, OCT4, SOX2, TRA-1-60, c-Myc, KLF4, LIN28, SSEA-4, SSEA-1, and combinations thereof.

A pluripotent stem cell marker can be detected by any detection method in the art. For example, a method for detecting a cell marker includes, but are not limited to, flow cytometry. For example, when flow cytometry is used as the detection method and a fluorescence-labeled antibody is used as the detection reagent, cells detected to have stronger fluorescence than the negative control (isotype control) may be determined to be “positive” for the marker. The proportion of cells positive for the detection reagent is often referred to as the “positive rate” herein. Any antibody known in the art may be used as a fluorescence-labeled antibody. For example, an antibody labeled with fluorescein isothiocyanate (FITC), phycoerythrin (PE), allophycocyanin (APC), or any combination thereof may be included, but is not limited thereto.

The proportion of pluripotent stem cells that constitute a cell aggregate can be determined, for example, by the positive rate for the pluripotent stem cell marker. The positive rate for the pluripotent stem cell marker in cells constituting a cell aggregate may be preferably 80% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more. A cell aggregate in which the proportion of cells expressing a pluripotent stem cell marker and/or the positive rate for the pluripotent stem cell marker is within the above-described numerical range is a highly undifferentiated and more homogeneous cell population.

The proportion of pluripotent stem cells may be determined by detecting the expression of one or more, two or more, or three or more pluripotent stem cell markers. In this case, the number of pluripotent stem cell marker within the above-described numerical range is not particularly limited. It may be, for example, one or more, two or more, three or more, or all of pluripotent stem cell markers detected.

### <<Suspension Culture>>

“Suspension culture” is one of the cell culture methods and refers to culturing cells in a liquid medium in a suspension state. As used herein, the “suspension state” refers to a state in which cells are not fixed by attachment or the like to an external matrix existing on the surface of a culture vessel (e.g., the inner surface such as the wall surface, the bottom surface, the lower surface of the cover, or the surface of the structure inside the culture vessel (e.g., a stirring blade)). The “suspension culture method” is a method of culturing cells by suspension culture. The cell in this method exists as aggregated cell masses in the culture solution. A method for suspending cells is not particularly limited, and includes stirring, rotation, and shaking.

In general, among cell culture methods, there is an adherent culture method as a culture method other than suspension culture. The “adherent culture” refers to culturing cells by attaching the cells to an external matrix or the like present on the surface of a culture vessel or the like. As an external matrix, for example, laminin, vitronectin, gelatin, collagen, E-cadherin chimeric antibody, and any combination thereof may be used but is not particularly limited thereto. In general, the pluripotent stem cells described above can be cultured not only by suspension culture but also by adherent culture.

### <<Medium and Mode of Medium Change>>

As used herein, the “medium” refers to a liquid or solid substance prepared for culturing cells. In principle, a medium contains at least the minimum necessary amount of components essential for growth and/or maintenance of a cell. Unless otherwise specified, the medium herein corresponds to a liquid medium for animal cells used for culturing animal-derived cells. As used herein, a liquid medium is often abbreviated simply as “medium.”

As used herein, the “basal medium” refers to a medium that is the basis for the media for various animal cell culture. Culture may be possible with a basal medium alone, but by adding various culture additives, it is also possible to prepare a medium according to the purpose, for example, a medium specific to various cells. A basal medium used herein includes, but is not particularly limited to, BME medium, BGJb medium, CMRL1066 medium, Glasgow MEM, Improved MEM Zinc Option medium, Iscove's Modified Dulbecco's Medium (IMDM), Medium 199, Eagle MEM, αMEM, Dulbecco's Modified Eagle's Medium (DMEM), Ham's F10 medium, Ham's F12 medium, RPMI 1640 medium, Fischer's medium, and mixed medium thereof (e.g., DMEM/F12 medium (Dulbecco's Modified Eagle's Medium/Nutrient Mixture F-12 Ham)). As DMEM/F12 medium, it is particularly preferable to use a medium obtained by mixing DMEM medium and Ham's F12 medium at a weight ratio in a range of 60/40 or more and 40/60 or less, for example, 58/42, 55/45, 52/48, 50/50, 48/52, 45/55, or 42/58. Other media such as those used for culturing human iPS cells and human ES cells may also be appropriately used.

Examples of preferable media for use in the present invention include media that do not contain serum, i.e., serum-free media.

As used herein, a “culture additive” is a substance other than serum and gaseous components added for culturing to a medium. Specific examples of a culture additive include, but are not limited to, L-ascorbic acid, insulin, transferrin, selenium, sodium hydrogen carbonate, growth factors, fatty acids or lipids, amino acids (e.g., non-essential amino acids), vitamins, cytokines, antioxidants, 2-mercaptoethanol, pyruvic acid, buffers, inorganic salts, antibiotics, and any combination thereof. Insulin, transferrin, and cytokines may be naturally occurring proteins isolated from tissues or serum of animals (e.g., humans, mice, rats, bovines, horses, and goats) or may be genetically engineered recombinant proteins. Further, as a growth factor, for example, basic fibroblast growth factor-2 (FGF2), transforming growth factor-β1 (TGF-β1), Activin A, IGF-1, MCP-1, IL-6, PAI, PEDF, IGFBP-2, LIF, IGFBP-7, and any combination thereof may be used, but is not limited thereto. An antibiotic, for example, penicillin, streptomycin, amphotericin B, or any combination thereof may be used, but is not limited thereto. FGF2 and/or TGF-β1 may be preferably used as a culture additive for the medium used in the present invention.

In addition, preferably, the medium contains a ROCK inhibitor. Examples of ROCK inhibitor include Y-27632. By containing a ROCK inhibitor in the medium, not-adhered state of pluripotent stem cells to a substrate or other cells and/or cell death under high shear stress can be remarkably suppressed.

Further, the medium preferably has a composition that does not contain LIF when a primed pluripotent stem cell is to be cultured. Preferably, the medium composition does not contain either one or both of a GSK3 inhibitor and/or an MEK/ERK inhibitor when a primed pluripotent stem cell is to be cultured. It is possible to culture a primed pluripotent stem cell while maintaining their undifferentiated state without making the primed pluripotent stem cells naïve in a medium that does not contain any of LIF, a GSK3 inhibitor, and an MEK/ERK inhibitor.

The medium used in the present invention may contain one or more culture additives described above. In general, examples of a medium to be supplemented with the culture additives include, but are not limited to, the basal medium.

A culture additive can be added to the medium in the form of a solution, a derivative, a salt, a mixed reagent, or the like. For example, L-ascorbic acid may be added to the medium in the form of a derivative such as magnesium ascorbate 2-phosphate. Selenium may be added to the medium in the form of a selenite salt (e.g., sodium selenite). In addition, insulin, transferrin, and selenium may also be added to the medium in the form of an insulin-transferrin-selenium (ITS) reagent. A medium supplemented with these culture additives, for example, a commercially available medium supplemented with at least one selected from L-ascorbic acid, insulin, transferrin, selenium, or sodium hydrogen carbonate may also be used. Examples of commercially available media supplemented with insulin and transferrin include CHO-S-SFM II (Life Technologies Japan Ltd.), Hybridoma-SFM (Life Technologies Japan Ltd.), eRDF Dry Powdered Media (Life Technologies Japan Ltd.), UltraCULTURE™ (BioWhittaker), UltraDOMA™ (BioWhittaker), UltraCHO™ (BioWhittaker), UltraMDCK™ (BioWhittaker), STEMPRO (registered trademark) hESC SFM (Life Technologies Japan Ltd.), Essential8™ (Life Technologies Japan Ltd.), StemFit (registered trademark) AK02N (AJINOMOTO CO., INC.), mTeSR1 (Veritas), and TeSR2 (Veritas).

A medium preferably used in the present invention includes, for example, a serum-free medium containing L-ascorbic acid, insulin, transferrin, selenium, and sodium hydrogen carbonate as culture additives in addition to at least one growth factor. As a culture additive, a serum-free DMEM/F12 medium containing L-ascorbic acid, insulin, transferrin, selenium, sodium hydrogen carbonate in addition to at least one growth factor (preferably FGF2 and TGF-β1) may also be appropriately used.

As used herein, the “mode of the medium change” refers to a method for supplying a medium, as a nutrient source, to cells for survival and growth of a cell and a method for removing a medium in which nutrients have been consumed by cells and metabolites have accumulated. The mode of the medium change includes, for example, and is not particularly limited to, the batch mode and the perfusion mode. The batch mode refers to replacing an arbitrary amount (e.g., whole amount, half amount) of the medium in the culture system (herein often referred to as “culture solution”) with a new medium at arbitrary culture intervals. The perfusion mode refers to continuous medium change by removing and supplying the medium in the culture system for a certain period. The amount of medium removed and supplied per unit time is referred to as the amount of medium perfused. Medium perfusion may be performed continuously or intermittently at multiple times.

### <<Gas Supply>>

As used herein, “gas supply” refers to supplying oxygen and carbon dioxide necessary for survival and/or growth or the like of a cell to the culture solution by gas aeration through the culture solution in which a cell is cultured. A component of gas used for gas supply include oxygen, nitrogen, carbon dioxide, and other gas components present in the atmosphere. Regarding the proportions of components in the gas supplied, the lower limit of oxygen is preferably 1%, 2%, 3%, 4%, 5%, 10%, or 20%, and the upper limit thereof is preferably 100%, 90%, 80%, 70%, 60%, 50%, 40%, 30%, or 20%. The lower limit of carbon dioxide is preferably 5%, 4%, 3%, 2%, 1%, or 0%, and the upper limit thereof is preferably 20%, 10%, 9%, 8%, 7%, 6%, or 5%. As the proportions of oxygen and carbon dioxide, any proportion may be selected independently to each other. For example, by adding nitrogen as a component other than oxygen and carbon dioxide, the oxygen concentration and the carbon dioxide concentration in the gas may be adjusted. In addition, the gas supplied may be prepared by mixing oxygen, carbon dioxide and nitrogen, each purified, or by mixing air with oxygen, carbon dioxide, or nitrogen. For example, the ratio of oxygen:carbon dioxide:nitrogen in the gas supplied includes, but are not limited to, 20:5:75, 20:4:76, 20:3:77, 20:2:78, 20:1:79, 20:0:80, 5:5:90, 5:0:95, 40:5:55, and 50:0:60. The ratio may not be constant during culturing and may be changed at any time. The gas supplied to the cell culture solution is preferably sterile and is, but is not limited to, preferably supplied to the culture solution through a filter. As used herein, the “carbon dioxide” may be referred to as “carbon dioxide gas,” and the “carbon dioxide concentration in the gas supplied” may be referred to as “carbon dioxide gas concentration.” In addition, the “carbon dioxide concentration in the liquid medium” may be referred to as “dissolved carbon dioxide gas concentration.”

### 1-3. Culture Step

The method in this aspect includes a suspension culture step and usually a subsequent culture step following passage. The method in this aspect may include a step of collecting a pluripotent stem cell population. Each of the steps will be described below.

### 1-3-1. Suspension Culture Step

The “suspension culture step” is a step of culturing cells to allow a pluripotent stem cell population to grow while maintaining their undifferentiated state. In the case of performing passage following this step, this step is specifically a step of culturing to grow a cell population before the step of passaging cells from suspension culture while maintaining their undifferentiated state. For suspension culture, an animal cell culture method known in the art may be used. For example, a suspension culture method in which cells are stirred in a liquid medium in a non-cell-adhesive vessel may be used.

### (Cells)

A cell used in this step is a cell capable of cell aggregation in suspension culture. As described in “1-2. Definitions of Terms” above for “pluripotent stem cell,” an animal cell, a human cell, and the like is preferable. In addition, a pluripotent stem cell such as an iPS cell and an ES cells may also be appropriately used. The pluripotent stem cell used in this step may be a single cell or a cell population (pluripotent stem cell population) consisting of a plurality of cells. When the pluripotent stem cell is a pluripotent stem cell population, the proportion (percentage) of cells expressing pluripotent stem cell markers (e.g., OCT4, SOX2, Nanog) and/or cells positive for pluripotent stem cell markers in the cell population is, for example, 90% or more, 91% or more, 92% or more, 93% or more, 94% or more, 95% or more, 96% or more, 97% or more, 98% or more, 99% or more, or 100% or less.

### (Culture Vessel)

The culture vessel used for suspension culture is not particularly limited, but a culture vessel treated to suppress protein adsorption on the inner surface of the vessel is preferable. For example, the shape of the culture vessel includes, but is not particularly limited to, dish-shaped, flask-shaped, well-shaped, bag-shaped, and spinner flask-shaped culture vessels. For example, 0.3c Single-Use Vessel (Eppendorf SE) may be used as a culture vessel.

The capacity of the culture vessel used may be appropriately selected and is not particularly limited. However, the lower limit of the volume capable of accommodating a medium and of culturing is preferably 1 mL, 2 mL, 4 mL, 10 mL, 20 mL, 30 mL, 50 mL, 100 mL, 200 mL, 500 mL, 1 L, 3 L, 5 L, 10 L, or 20 L, and the upper limit thereof is preferably 100 L, 50 L, 20 L, 10 L, 5 L, 3 L, 1 L, 500 mL, 200 mL, 100 mL, 50 mL, or 30 mL. When a stirring blade-type reactor of arbitrary capacity is used, the capacity may be within the range of the working volume specified by each manufacturer of the reactor.

As used herein, the volume of the medium that is being accommodated in the culture vessel and used for cell culture is referred to as the culture volume or the amount of the culture solution.

### (Medium)

The medium used for suspension culture is a medium comprising a basal medium, as described above in “1-2. Definitions of Terms,” containing a ROCK inhibitor. The medium is not limited as long as it contains a ROCK inhibitor and can proliferate and/or maintain pluripotent stem cells. It is particularly preferable to use a medium that does not contain leukemia inhibitory factor.

The culture additive composition of the medium used in this step may not be constant. Specifically, the culture additive composition of the medium at the start of culture in this step may be different from the culture additive composition of the medium used for medium change by the perfusion mode during culture in this step. A plurality of types of media may be used for medium change by the perfusion mode during culture in this step, and the medium used for medium change by the perfusion mode may be switched to one with a different culture additive composition at arbitrary time during culture. In addition, the culture additive composition of a liquid medium used in perfusion may be changed during culture. Changing the culture additive composition of the medium as described above makes it possible to continuously control the concentration of arbitrary culture additive or medium component in the culture system in accordance with various amount of medium perfused per unit time, thereby achieving an appropriate concentration transition.

For example, the ROCK inhibitor may have a lower limit of 1 μM, 2 μM, 3 μM, 5 μM, 7 μM, or 10 μM as the final concentration in the liquid medium at the start of culture in this step.

The upper limit of the ROCK inhibitor concentration in the liquid medium at the start of culture in this step is not particularly limited, and may be determined depending on conditions, including a range that does not cause cell death, a range that does not cause deviation from the undifferentiated state, and the solubility of the ROCK inhibitor.

For example, the ROCK inhibitor in the liquid medium in the perfusion mode of this step may have an upper limit of 50 μM, 40 μM, 30 μM, or 20 μM as the final concentration in the liquid medium at the start of culture.

Although not particularly limited, the concentration of the ROCK inhibitor in the liquid medium used for medium change by the perfusion mode in this step is preferably lower than the concentration of the ROCK inhibitor in the liquid medium at the start of culture in this step.

The upper limit of the ROCK inhibitor concentration as the final concentration in the liquid medium used for medium change by the perfusion mode in this step is not particularly limited, and may be determined depending on conditions, including a range that does not cause cell death, a range that does not cause deviation from the undifferentiated state, and the solubility of the ROCK inhibitor.

For example, the ROCK inhibitor may have an upper limit of 50 μM, 40 μM, 30 μM, or 20 μM as the final concentration in the liquid medium used for medium change by the perfusion mode in this step.

For example, the ROCK inhibitor may have a lower limit of 0 μM, 1 μM, 2 μM, 3 μM, 5 μM, 7 μM, or 10 μM as the final concentration in the liquid medium used for medium change by the perfusion mode in this step.

The method of adding the ROCK inhibitor is not particularly limited as long as the concentration of the ROCK inhibitor in the medium is within the above-described range. For example, the concentration may be adjusted by directly adding the ROCK inhibitor to the medium in a total amount that falls within the concentration range or by adding a ROCK inhibitor solution diluted with a different solvent, which is mixed with the medium.

In the present invention, the volume of carbon dioxide gas supplied may be reduced in line with the progress of culture. In other words, the dissolved carbon dioxide gas concentration in the culture solution may be reduced. Meanwhile, when performing medium change by the perfusion described above, if the dissolved carbon dioxide gas concentration in the medium used for perfusion is higher than the dissolved carbon dioxide gas concentration in the culture solution, the dissolved carbon dioxide gas concentration in the culture solution would increase. Therefore, the dissolved carbon dioxide gas concentration in the medium used for perfusion is preferably lower than the dissolved carbon dioxide gas concentration in the culture solution.

### (Seeding Density)

When performing suspension culture by the perfusion mode, the density of cells to be seeded in a new medium (seeding density) may be appropriately adjusted, considering the state of cells used for seeding, the culture time in this step, and the number of cells required after culture. Although not limited, in general, the lower limit thereof may be in a range of 0.01×10⁵ cells/mL, 0.1×10⁵ cells/mL, 1×10⁵ cells/mL, or 2×10⁵ cells/mL, and the upper limit thereof may be in a range of 20×10⁵ cells/mL or 10×10⁵ cells/mL. The growth efficiency, especially in the early stage of culture, depends on the seeding density. Therefore, for example, the lower limit of the seeding density is preferably 1×10⁵ cells/mL, and the upper limit thereof is preferably 5×10⁵ cells/mL.

### (Culture Conditions)

Culture conditions such as culture temperature, culture time, CO₂ concentration, and O₂ concentration are not particularly limited. Culture may be carried out within the range in conventional methods in the art. For example, the lower limit of the culture temperature may be 20° C. or 35° C., and the upper limit thereof may be 45° C. or 40° C., but preferably, the culture temperature is 37° C. In addition, for example, the lower limit of the culture time for one passage period may be 0.5 hours or 6 hours, and the upper limit thereof may be 192 hours, 120 hours, 96 hours, 72 hours, or 48 hours. For example, the lower limit of the CO₂ concentration during culture may be 0%, 0.5%, 1%, 2%, 3%, 4%, or 5%, and the upper limit thereof may be 10% or 5%, and more preferably, the CO₂ concentration during culture is 5%. For example, the lower limit of the O₂ concentration during culture may be 3% or 5%, and the upper limit thereof may be 21% or 20%, and more preferably, the 02 concentration during culture is 21%.

### (Culture Method)

In the suspension culture of this step, the medium during culture is in a flowing state. The “flow culture” refers to culturing under conditions that allow the medium to flow. In the case of flow culture, a method that allows the medium to flow to promote cell aggregation is preferable. For example, as such a culture method, the rotation culture method, the rocking culture method, the stirring culture method, and any combination thereof are included.

The “rotation culture method” (including the shaking culture method) refers to a method for performing culture under conditions that allow a medium to flow such that cells gather at one point due to the stress (force) (centrifugal force, centripetal force) caused by rotational flow. Specifically, culture is performed by rotating a culture vessel accommodating a medium containing cells along a substantially horizontal plane in a closed orbit such as a circle, an ellipse, a deformed circle, a deformed ellipse, or the like.

The rotation speed is not particularly limited, but the lower limit thereof may be 1 rpm or more, 10 rpm or more, 50 rpm or more, 60 rpm or more, 70 rpm or more, 80 rpm or more, 83 rpm or more, 85 rpm or more, or 90 rpm or more. Meanwhile, the upper limit thereof may be 200 rpm or less, 150 rpm or less, 120 rpm or less, 115 rpm or less, 110 rpm or less, 105 rpm or less, 100 rpm or less, 95 rpm or less, or 90 rpm or less. The amplitude of a shaker used for rotation culture is not particularly limited, but the lower limit thereof may be, for example, 1 mm or more, 10 mm or more, 20 mm or more, or 25 mm or more. Meanwhile, the upper limit thereof may be, for example, 200 mm or less, 100 mm or less, 50 mm or less, 30 mm or less, or 25 mm or less. The radius of rotation during rotation culture is not particularly limited, but the amplitude is preferably set within the above-described range. The lower limit of the radius of rotation may be, for example, 5 mm or more or 10 mm or more, and the upper limit of the radius of rotation may be, for example, 100 mm or less or 50 mm or less. In particular, when the present method is used as a method for producing a cell aggregate or the like, it is preferable to set the rotation condition within the above-described range since a cell aggregate having an appropriate size may be easily produced.

The “rocking culture method” refers to a method for performing culture under conditions that impart a rocking flow to a medium by linear reciprocating motion, such as rocking stirring. Specifically, culture is performed by rocking a culture vessel accommodating a medium containing cells along a plane substantially perpendicular to the horizontal plane. The rocking rate is not particularly limited. For example, given that one round trip is regarded as one time, the lower limit thereof may be two or more times, four or more times, six or more times, eight or more times, or ten or more times of rocking per minute. Meanwhile, the upper limit thereof may be 15 times or less, 20 times or less, 25 times or less, or 50 times or less of rocking per minute. During rocking, it is preferable to give the culture vessel a slight angle, namely a rocking angle, relative to the vertical plane. The rocking angle is not particularly limited. For example, the lower limit thereof may be 0.1° or more, 2° or more, 4° or more, 6° or more, or 8° or more, and the upper limit thereof may be 200 or less, 180 or less, 150 or less, 120 or less, or 10° or less. When the present method is used as a method for producing a cell aggregate or the like, it is preferable to set the rocking condition within the above-described range since a cell aggregate having an appropriate size may be easily produced.

Further, culture may also be performed while stirring by a combination of the above-described rotation and rocking motions.

The “stirring culture method” refers to a method for culturing while stirring a culture solution with a stirring blade or stirrer and under conditions that allow cells and/or cell aggregates and the like to be dispersed in the culture solution. When the medium during culture is made fluid by stirring with a stirring blade, although not particularly limited, the lower limit of the stirring speed is preferably 1 rpm, 5 rpm, 10 rpm, 20 rpm, 30 rpm, 40 rpm, 50 rpm, 60 rpm, 70 rpm, 80 rpm, 90 rpm, 100 rpm, 110 rpm, 120 rpm, or 130 rpm, and the upper limit thereof is preferably 200 rpm, 190 rpm, 180 rpm, 170 rpm, 160 rpm, 150 rpm, 140 rpm, 130 rpm, 120 rpm, 110 rpm, 100 rpm, 90 rpm, 80 rpm, 70 rpm, 60 rpm, 50 rpm, 40 rpm, or 30 rpm.

In the “stirring culture method,” which is a suspension culture in a stirring mode using a reactor with a stirring blade or the like, it is preferable to control the shear stress applied to the cells during culture. Animal cells, including pluripotent stem cells, are generally more susceptible to physical stress than other types of cells. Therefore, when excessive shear stress is applied to cells during stirring culture, cells might be physically damaged, their growth ability is reduced, and in the case of pluripotent stem cells, they cannot maintain their undifferentiated property.

Shear stress applied to cells in stirring culture is not limited, but depends on, for example, the blade tip speed. The blade tip speed is the peripheral speed of the tip of the stirring blade and can be obtained as the following formula: Blade diameter [m]×Circumference ratio×Rotational rate [rps]=Blade tip speed [m/s]. In a case in which a plurality of blade diameters are obtained because of the tip shape of the stirring blade, the largest diameter may be used.

The blade tip speed is not particularly limited, but the lower limit thereof is preferably 0.05 m/s, 0.08 m/s, 0.10 m/s, 0.13 m/s, 0.17 m/s, 0.20 m/s, 0.23 m/s, 0.25 m/s, or 0.30 m/s. By setting the blade tip speed within this range, excessive aggregation of cells may be suppressed while maintaining pluripotent stem cells undifferentiated.

Further, the blade tip speed is not particularly limited, but the upper limit thereof is preferably 1.37 m/s, 1.00 m/s, 0.84 m/s, 0.50 m/s, 0.42 m/s, 0.34 m/s, or 0.30 m/s. By setting the blade tip speed within this range, the flowing state of the medium in the culture system may be stabilized while maintaining pluripotent stem cells undifferentiated.

It is preferable to start the medium change by the perfusion mode in a state in which the cells seeded in the culture solution adhere to each other and form aggregates. When medium change is performed by the perfusion mode, for example, it is preferable to start perfusion in the perfusion mode after the formation of cell aggregates. As a result, cell aggregates can be retained in the culture solution upon medium change using a filter that removes only the medium but not cells from the culture solution, which will be described later. All the cells in the culture solution do not need to form aggregates, and cells in a single cell state may exist. Cells in a single cell state at the start of perfusion may form cell aggregates under medium perfusion. The proportion of the number of cells forming aggregates to the seeded cell count at the start of medium perfusion is not particularly limited, but the lower limit thereof is preferably 10%, 20%, 30%, 40%, 50%, 60%, 70%, or 80%, and the upper limit thereof is preferably 300%, 200%, 150%, 140%, 130%, 120%, 110%, 100%, 90%, or 80%. In general, some of the cells seeded in the suspension culture die, and the cell count temporarily decreases relative to the seeding concentration. However, the proportion of this decrease is preferably low. When medium change is performed by the perfusion mode, there is a concern that the excessively high proportion of the number of cells forming aggregates to the seeded cell count at the start of perfusion may result in progressing of nutrient depletion before the start of perfusion and cells being adversely affected. Therefore, the proportion is preferably not excessively high. For this reason, it is preferable to set the lower limit to 50% and the upper limit to 150% for the range of the proportion of the number of cells forming aggregates to the seeded cell count.

In addition, the timing to start the medium change by the perfusion mode may be set arbitrarily as long as the cells in the culture solution are in a state of adhering to each other and forming aggregates. Although not particularly limited, the timing to start perfusion is preferably, for example, 72 hours or later, 60 hours or later, 48 hours or later, 42 hours or later, 36 hours or later, 30 hours or later, 24 hours or later, 18 hours or later, or 12 hours or later after seeding cells and starting culture.

The amount of medium perfused per unit time at the start of perfusion (herein sometimes referred to as “reference perfusion rate”) may be determined arbitrarily. The reference perfusion rate is an amount of medium perfused obtained by multiplying the amount of medium perfused for substituting 100% of the medium volume in a certain period by a start coefficient based on the culture conditions at the start of culture. In this connection, the length of the certain period is not particularly limited, and may be, for example, 1 hour, 3 hours, 5 hours, 6 hours, 9 hours, 12 hours, 15 hours, 18 hours, 20 hours, 24 hours, 30 hours, 36 hours, 42 hours, 48 hours, 60 hours, or 72 hours.

Although not particularly limited, for example, when the certain period is 24 hours, the reference perfusion rate can be set based on a value obtained by multiplying the culture volume by the proportion of the unit time length to 24 hours. Specifically, for example, when the unit time length is 1 hour and the certain period is 24 hours, the reference perfusion rate is based on a value obtained by dividing the culture volume by 24.

A value obtained by multiplying the above value by an appropriate value according to the culture conditions at the start of culture as the start coefficient, such as the cell seeding density and the proportion of the number of cells forming aggregates to the seeded cell count at the start of perfusion, may be determined as the reference perfusion rate. The lower limit of the start coefficient is preferably 0.1, 0.5, 0.6, 0.7, 0.8, 0.9, or 1.0, and the upper limit thereof is preferably 2.0, 1.9, 1.8, 1.7, 1.6, 1.5, 1.4, 1.3, 1.2, 1.1, or 1.0.

The start coefficient can be appropriately set according to the purpose and conditions. For example, with a start coefficient of a particular condition set at 1.0, a value based on the magnitude of deviation from that particular condition (e.g., the proportion of the actual cell density to the certain cell seeding density or the ratio of an actual proportion of the number of cells forming aggregates to the seeded cell count at the start of perfusion to a certain proportion of the number of cells forming aggregates to the seeded cell count at the start of perfusion) may be used as the start coefficient. Examples of the particular condition include a standard culture condition for using allogeneic cells and a culture condition recommended by the cell provider.

After starting perfusion, the timing to start the control of the amount of medium perfused using the method of the present invention may be set arbitrarily. The control of the amount of medium perfused may be started at the start of perfusion or 6 hours or later, 12 hours or later, 18 hours or later, or 24 hours or later after medium perfusion. It is preferable to start the control of the amount of medium perfused before the culture environment, such as the lactic acid concentration and pH, changes remarkably, to an extent that cells begin to be affected adversely.

The amount of medium perfused per unit time of medium change by the perfusion mode (herein sometimes referred to as “variable perfusion rate”) preferably has a lower limit of 1%, 3% 4%, 5%, 10%, 20%, 30%, 40%, or 50% and an upper limit of 100%, 90%, 80%, 70%, 60%, or 50% with respect to the culture volume. In this case, the “amount of medium perfused per unit time” refers to the amount of medium perfused per hour.

It is preferable to control the variable perfusion rate in line with the progress of culture in the above-described range. In other words, it is preferable to control the variable perfusion rate in a range of 1% to 100% of the culture volume based on the reference perfusion rate and the culture variable in a particular culture condition in the suspension culture step. As long as the variable perfusion rate is controlled by the method of the present invention, its transition is arbitrary. For example, perfusion may be performed at a constant amount in unit time, or the amount of medium perfused may be decreased in the first half of unit time and increased in the second half of the unit time. Intermittent perfusion may be performed by stopping perfusion only for a part of the unit time. Preferably, the control of the variable perfusion rate in line with the progress of culture is based on one or more culture variables. The culture variable is a variable based on a particular culture condition. Specific examples of the culture variable include the cell density, the cell count, the cell aggregate size or volume, the amount of lactic acid in the culture solution, pH in the culture solution, and the amount of lactic acid produced by metabolism per cell per unit time. In addition, the cell density increasing rate, which is the proportion of a cell density relative to a cell density at the start of controlling the amount of medium perfused, may be set as a culture variable. The aggregate volume increasing rate, which is the proportion of a cell aggregate volume relative to a cell aggregate volume at the start of controlling the amount of medium perfused, may also be set as a culture variable. For example, when one of the culture variables is the cell density increasing rate, the amount of medium perfused may be controlled by increasing the variable perfusion rate based on the increase of the cell density increasing rate. In addition, for example, when one of the culture variables is the aggregate volume increasing rate, the amount of medium perfused may be controlled by increasing the variable perfusion rate based on the increase of the aggregate volume increasing rate.

The amount of medium perfused per unit time may be changed continuously or intermittently in in accordance with the changes in one or more of these culture variables. For example, the amount of medium perfused per unit time may be controlled to be proportional to one or more culture variables, respectively. In other words, if it is based on a plurality of culture variables, the amount of medium perfused may be controlled such that a proportional relationship is established for each culture variable when the other culture variables are constants.

For example, when the cell density is a culture variable, the amount of medium perfused per unit time may be increased in line with the increase of the cell density. For example, it may be increased proportionally with the increase of the cell density. When pH is a culture variable, the amount of medium perfused per unit time may be controlled to suppress the decrease in pH. Suppressing the decrease in pH is maintaining or slightly increasing pH to prevent a pH decrease or decelerating the pH decreasing rate. It is possible to suppress the decrease in pH by increasing the amount of medium perfused and/or decreasing the amount of carbon dioxide gas supplied to the medium as described later. Therefore, the pH decrease may be suppressed by, for example, increasing the amount of medium perfused per unit time based on the pH decrease.

Hereinafter, the control of the amount of medium perfused will be described using mathematical formulas, taking as an example the case in which the cell density or the like is used as the culture variable. However, this is only an example, and even when other information is used as the culture variable, the amount of medium perfused can be controlled in the same manner.

For example, in a case in which one of the culture variables is the cell density increasing rate, given that the amount of medium perfused at the beginning of controlling the amount of medium perfused per unit time (i.e., reference perfusion rate) is F₀, the cell density at that time is C₀, and the cell density at an arbitrary time point in each subsequent culture time is C, the amount of medium perfused per unit time at the arbitrary time point (i.e., variable perfusion rate) F can be expressed by the following Formula 1 that is proportional to the cell density increasing rate.

$$F = {F}_{0} \times \frac{C}{{C}_{0}} \qquad [ Formula 1 ]$$

The value of C may be a previously assumed value or reflect a value measured during culture. It may be switched during culture, such as by using the assumed value as C in the first half of culture and applying the measured value as C in the second half of culture.

The cell density may also be replaced by the cell count or the cell aggregate size or volume. For example, in a case in which the cell density is set to be the cell aggregate volume (C being the cell aggregate volume at an arbitrary time point during culture and C₀ being the cell aggregate volume at the start of the control), F (variable perfusion rate) when one of the culture variables is the cell aggregate volume increasing rate can be expressed by Formula 1 that is proportional to the cell aggregate volume increasing rate. The following equation 2 can be obtained by multiplying Formula 1 above by M as a correction coefficient for correcting the difference in cell characteristics or the like due to the cell line and the culture history of the cell line.

$$F = M \times {F}_{0} \times \frac{C}{{C}_{0}} \qquad [ Formula 2 ]$$

The difference in cell characteristics is not limited but includes resistance to lactic acid in the culture solution. It may be set to reflect the upper limit of the lactic acid concentration that does not have remarkable adverse effects on cells. Although not particularly limited, for example, the correction coefficient M may be regarded as a value representing the difference in resistance to a stringent culture environment due to the cell line. As the absolute value of the correction coefficient M, the lower limit is preferably 0.1, 0.2, 0.3, 0.4, 0.5, 0.6, 0.7, 0.8, 0.9, or 1.0, and the upper limit is preferably 2.0, 1.9, 1.8, 1.7, 1.6, 1.5, 1.4, 1.3, 1.2, 1.1, or 1.0. The value of M may be positive or negative. For example, when the cell density, cell density increasing rate, cell count, or cell aggregate size or volume is used as culture variables, a positive M value is used because, generally, it is preferable to increase the amount of medium perfused as these variables increase. Meanwhile, for example, when pH is used instead of the cell density as a culture variable, a negative M value is used because, generally, it is preferable to increase the amount of medium perfused as the pH decreases.

As the value of the correction coefficient M, the value of the lactic acid resistance in the culture solution of the cell line used, with the lactic acid resistance in the culture solution of a certain human iPS cell line (e.g., cell line 1231A3 or 1383D6) set to 1.0, may be set. The lactic acid resistance of the cell line used may be determined based on, for example, an IC₅₀ value calculated from the culture with lactic acid being added or based on accumulated lactic acid concentrations before and after the cell growth began to decline in experimental culture. Information on lactic acid resistance may be provided by the provider of the cell line or obtained by actual measurement.

In addition, as the value of the correction coefficient M, a value that indicates resistance to low pH in the culture solution may also be used. In this case, for example, the value of M may also be set by reflecting the optimum pH of the cell line used or the lower limit of carbon dioxide gas concentration, which will be described later. In general, the higher the optimum pH and/or the lower the lower limit for adjusting the carbon dioxide gas concentration, the smaller the value of M may be. As the value of the correction coefficient M, the value of the pH resistance in the culture solution of the cell line used, with the pH resistance in the culture solution of a specific human iPS cell line set to 1.0, may be set. Information on pH resistance may be provided by the provider of the cell line or obtained by actual measurement.

It is also possible to multiply Formula 2 above by a variable K that varies depending on the amount of lactic acid produced by metabolism per cell per unit time to obtain the following Formula 3.

$$F = M \times K \times {F}_{0} \times \frac{C}{{C}_{0}} \qquad [ Formula 3 ]$$

K can be expressed by the following Formula 4 with the amount of lactic acid produced by metabolism per cell per unit time at a certain time point, L₀, and the amount of lactic acid produced by metabolism per cell per unit time in each subsequent culture time, L.

$$K = \frac{L}{{L}_{0}} \qquad [ Formula 4 ]$$

In this regard, the amount of lactic acid produced by metabolism per cell per unit time at a certain time point refers to a value obtained by dividing a change in the amount of lactic acid in the culture solution per unit time until the certain time point by the average cell count within the unit time (i.e., a value obtained by dividing a change in the lactic acid concentration in the culture solution per unit time until the certain time point by the average cell density within the unit time). In this connection, as the amount of lactic acid or lactic acid concentration in the culture solution, for example, a value directly measured in the culture solution, a value measured in a sample collected in a small amount from the culture solution, or a value measured in the medium removed from the culture system by perfusion may be used.

The upper limit of the lactic acid concentration when the amount of medium perfused is started to be changed is preferably 10 mM, 9 mM, 8 mM, or 7 mM. When it is desirable to control the lactic acid concentration, if the lactic acid concentration when the amount of medium perfused is started to be changed was high, the amount of medium used for perfusion increases in general.

The amount of lactic acid produced by metabolism per cell per unit time may differ between cell lines or depending on culture conditions or the like. Therefore, it is preferable to measure and confirm the amount in advance in accordance with the cells to be used. The lower limit thereof is preferably 1.0×10⁻¹⁰ mmol/cell/h, 3.0×10⁻¹⁰ mmol/cell/h, 5.0×10⁻¹⁰ mmol/cell/h, 7.0×10⁻¹⁰ mmol/cell/h, 1.0×10⁻⁹ mmol/cell/h, 1.1×10⁻⁹ mmol/cell/h, 1.2×10⁻⁹ mmol/cell/h, or 1.3×10⁻⁹ mmol/cell/h. The upper limit thereof is preferably 2.5×10⁻⁹ mmol/cell/h, 2.0×10⁻⁹ mmol/cell/h, 1.9×10⁻⁹ mmol/cell/h, 1.8×10⁻⁹ mmol/cell/h, 1.7×10⁻⁹ mmol/cell/h, 1.6×10⁻⁹ mmol/cell/h, 1.5×10⁻⁹ mmol/cell/h, 1.4×10⁻⁹ mmol/cell/h, or 1.3×10⁻⁹ mmol/cell/h. Preferably, the lactic acid production rate in the suspension culture step is maintained between the lower and upper limits described above. It is also possible to assume a change in the amount of lactic acid produced by metabolism per cell per unit time during culture based on the HK2 gene expression level and change thereof.

It is preferable to control the amount of medium perfused during culture according to the above-described formulas in principle. However, applying the above-described formulas may be temporarily suspended, and the lactic acid concentration and pH in the culture solution may be restored to their assumed ranges by increasing or decreasing by arbitrary amount, or maintaining the amount of medium perfused in a case in which the lactic acid concentration and pH in the culture solution and the like measured by arbitrary methods are deviated from the initially assumed ranges, namely the case in which they are out of the ranges of values that do not adversely affect cells, or in a case in which they are within the ranges of values that do not adversely affect cells but are continuously out of the initially assumed ranges, and perfusion with an extra amount of medium is required. The assumed ranges may be appropriately determined according to conditions such as the cost and equipment. Preferably, the assumed ranges are set within the ranges of the lactic acid concentration and pH that do not adversely affect cells. Examples of the upper limit of the lactic acid concentration that does not adversely affect cells include 20 mM, 18 mM, 16 mM, 14 mM, 12 mM, 10 mM, and 8 mM, but are not particularly limited thereto because it may vary depending on the cell line or the like. Examples of the lower limit of pH that does not adversely affect cells include 6.5, 6.6, 6.7, 6.8, 6.9, and 7.0. Preferably, the pH in the suspension culture step or at the start of control is maintained at the lower limit described above or more. Meanwhile, preferably, the lactic acid concentration in the culture solution in the suspension culture step or at the start of control is maintained at the upper limit described above or less.

After the cell density reached 8.0×10⁵ cells/mL, the degree of susceptibility to changes in the culture environment increases. Therefore, it is preferable to control the amount of medium perfused such that the total amount of medium used for medium change for any 6 hours of culture after the cell density reached 8.0×10⁵ cells/mL becomes greater than the total amount of medium used for medium change for 6 hours of culture immediately before the any 6 hours of culture. In other words, the control of the amount of medium perfused includes increasing an amount of medium perfused for any 6 hours of culture after the cell density of the pluripotent stem cells reached 8.0×10⁵ cells/mL compared to an amount of medium perfused for 6 hours of culture immediately therebefore.

The medium change by the perfusion mode may be performed by continuously suctioning the culture solution from which the cells have been separated by a filter or the like from the vessel while continuing the culture and continuously feeding a new medium. The opening size of the filter to be used may be smaller than cell aggregates. In addition, the size may be such that dead cells or the like in the culture solution can pass through. The lower limit is, but is not particularly limited to, preferably 0.1 m, 1 m, 5 m, 10 m, or 20 m, and the upper limit is, but is not particularly limited to, preferably 40 μm, 35 μm, 30 μm, 25 μm, 20 μm, or 15 μm.

In the suspension culture step by this perfusion mode, the cell count obtained through growth can be set arbitrarily. The cell count and state of interest may be appropriately determined according to the type of cells to be cultured, the purpose of cell aggregation, the type of medium, and the culture conditions. For example, the degree of cell growth is not particularly limited, but the lower limit may be 2 times, 3 times, 4.5 times, 5 times, 6 times, 6.5 times, 7 times, 8 times, 9 times or 10 times compared to the cell seeding concentration at the start of culture. Meanwhile, the upper limit is not particularly set but may be, for example, 100 times, 50 times, 40 times, 30 times, 20 times, or 10 times. In particular, cells preferably grow 10 times or more. The degree of cell growth may be measured, for example, on day 1 of culture, day 2 of culture, day 3 of culture, day 4 of culture, day 5 of culture, day 6 of culture, or later. In addition, measurements may be taken for a plurality of times on different days.

In the suspension culture step in this perfusion mode, it is possible to isolate a part of the pluripotent stem cells during culture and to confirm the cell count and the cell aggregate size. An aggregate of pluripotent stem cells taken out during culture may be loosened into single cells by, for example, enzymatic treatment, and the viable cell count may be measured by a method such as the trypan blue method. Alternatively, it is also possible to estimate the cell count from the number and size of aggregates of pluripotent stem cells taken out during culture. In addition, it is not particularly limited but the cell aggregate size or the cell aggregate volume can be measured by size measurement using a laser method, a method for acquiring an image and calculating the size from the image, or the like.

A pluripotent stem cell population produced in this suspension culture step may comprise a cell aggregate. The cell aggregate size is not particularly limited, but the average diameter of the maximum width size in an observation image of cell aggregates in the same culture system when observed with a microscope may be the lower limit of 40 μm, 50 μm, 60 μm, 70 μm, 80 μm, 90 μm, or 100 μm or, the upper limit of 500 μm, 400 μm, 300 μm, 250 μm, 200 μm, or 150 μm. Cell aggregates within this range are preferable as a growth environment for cells because oxygen and nutrients are easily supplied to the cells inside. Particularly preferably, the cell aggregate size has a lower limit of 40 μm and an upper limit of 250 μm. A pluripotent stem cell population, such as a cell aggregate, may be collected in the suspension culture step. A method for collecting a pluripotent stem cell population, such as a cell aggregate, may follow a conventional method used in cell culture methods in the art, which is not particularly limited.

In the pluripotent stem cell population produced in this suspension culture step has a lower limit of 30%, 40%, 50%, 60%, 70%, 75%, 80%, 85%, 90%, 95%, 98%, or 100% of the cell aggregates on the weight basis is preferably within the above-described size.

In terms of increasing the production efficiency in one batch, the lower limit of the cell density at the end of culture in this suspension culture step is preferably 1.0×10⁶ cells/mL, 2.0×10⁶ cells/mL, or 3.0×10⁶ cells/mL.

In this suspension culture step in the perfusion mode, the medium removed from the culture system in the perfusion mode may be used for measuring the concentrations of nutrients and metabolites in the medium. For example, although not limited, it is possible to measure the glucose concentration, lactic acid concentration, or the like in the removed medium using a medium component measuring device using an enzymatic electrode reaction. The information above may be reflected in the control of amount of medium perfused.

The glucose concentration in the medium removed from the culture system by the perfusion mode preferably has a lower limit of 1 mM, 2 mM, 3 mM, 4 mM, 5 mM, 6 mM, 7 mM, 8 mM, 9 mM, or 10 mM and an upper limit of 20 mM, 19 mM, 18 mM, 17 mM, 16 mM, 15 mM, 14 mM, 13 mM, 12 mM, or 11 mM, but are not particularly limited. For example, the lower limit may be 4 mM, and the upper limit may be 16 mM. In addition, the lactic acid concentration in the medium removed from the culture system by the perfusion mode preferably has a lower limit of 0 mM, 1 mM, 2 mM, 3 mM, 4 mM, 5 mM, 6 mM, 7 mM, 8 mM, 9 mM, or 10 mM and an upper limit of 20 mM, 19 mM, 18 mM, 17 mM, 16 mM, 15 mM, 14 mM, 13 mM, 12 mM, or 11 mM. For example, the lower limit may be 0 mM, and the upper limit may be 12 mM.

In the suspension culture of this step, any method to supply gas may be used, and a standard technique used in general culture methods may be used. For example, but not limited to, the gas supplied may be supplied by aerating the liquid surface of the culture solution, or it may be bubbled in the culture solution using a sparger. However, a method to aerate the liquid surface of the culture solution is more preferable.

Regarding the amount of gas supplied, when cells are cultured in a culture apparatus such as an incubator, the amount of gas supplied may be an amount sufficient to fill the inside of the apparatus. When cells are cultured in a vessel such as a bioreactor, aeration is performed via a gas supplying port of the vessel, and the amount of gas supplied may be appropriately determined considering the culture volume, the surface area of the culture solution, the gas requirement of the culture cells, the speed of gas movement in the culture solution, and the like. As an example, in the case of culture with a culture solution amount of 142 mL using a 0.3c Single-Use Vessel (Eppendorf SE), a suitable amount of gas supplied is 3 L/h. When the culture solution amount is increased relative to the culture solution amount described above, the amount of gas supplied may be increased. When the culture solution amount is decreased relative to the culture solution amount described above, the amount of gas supplied may be decreased.

In this suspension culture step, the concentration of carbon dioxide gas supplied to the liquid medium can be changed. In a general cell culture method, the gas concentration, such as a carbon dioxide gas concentration, is constant throughout the culture. However, it must be changed appropriately to respond to the sequential changes in the cell state and the medium environment during culture. In this step, the carbon dioxide gas concentration in the gas supplied preferably has a lower limit of 0%, 1%, 1.5%, 2%, or 2.5% and an upper limit of 10%, 9%, 8%, 7%, 6%, or 5%. The amount of carbon dioxide gas supplied in the liquid medium is obtained by multiplying the carbon dioxide gas concentration in the gas supplied by the supply amount of the gas supplied. In other words, as the method for changing the amount of carbon dioxide gas supplied in the culture solution, a method for changing the carbon dioxide gas concentration in the gas supplied, a method for changing the supply amount of the gas supplied containing carbon dioxide gas, or a method in which both methods are combined may be used.

It is believed that as the culture progresses, the cells grow, the total volume of oxygen consumed increases, and carbon dioxide is released by the cells themselves. Therefore, the culture environment can be more uniformly controlled by altering the supply amount of carbon dioxide gas from the outside. In addition, the influence of metabolites other than carbon dioxide discharged by the cells in line with the progress of culture on the culture environment can be controlled by reducing the supply amount of carbon dioxide gas supplied. In other words, it is preferable to reduce the supply amount of carbon dioxide gas supplied in line with the progress of culture in this step. For example, it is preferable to reduce the carbon dioxide gas concentration in line with the progress in culture when the supply amount of the gas supplied is constant. The amount of carbon dioxide gas supplied does not need to be reduced monotonously, and may be gradually reduced by increasing or decreasing the carbon dioxide gas concentration to adjust the balance.

For example, the average carbon dioxide gas concentration for a certain length of time can be reduced to be equal to or less than the average carbon dioxide gas concentration for the previous same length of time. In this case, the length of time is not particularly limited but may be, for example, 0.5 hours, 1 hour, 1.5 hours, 2 hours, 2.5 hours, 3 hours, 4 hours, 5 hours, 6 hours, 8 hours, 12 hours, 18 hours, or 24 hours. Alternatively, the length of time may be, for example, 1 hour to 6 hours or 2 hours to 4 hours. The time interval separated by this time may be fixed in position within the culture time (e.g., the interval from 0 hours to 6 hours after the start of culture) or may be set according to the time point of interest (e.g., the 6 hours immediately before the time point of interest).

There is no particular limitation on the rate of decrease of the carbon dioxide gas concentration. For example, the carbon dioxide gas concentration is reduced to a range of 0% to 5%, 0% to 4%, 0% to 3%, or 0% to 2.5% after a certain period from the start of the reduction. For example, it may be reduced to the above-described range 0.5 days or later, 1 day or later, or 1.5 days or later after the start of reduction. As the carbon dioxide gas concentration, for example, the average of the carbon dioxide gas concentration over a certain length of time as described above may be used. The length of time used herein may be selected independently of the length of time described above. For example, the average carbon dioxide gas concentration for 2 to 4 hours may be reduced to be no more than the average carbon dioxide gas concentration for the previous same length of time. As a result, the average of the carbon dioxide gas concentration for 6 hours can be reduced to a range of 0% to 2.5% after one or more days from the start of reduction.

The amount of carbon dioxide gas supplied to the liquid medium may be altered based on one or more indicators. Examples of indicators for decreasing the carbon dioxide gas concentration in line with the progress of culture include pH, cell density, lactic acid concentration, and lactic acid production rate of cells. These indicators may be selected independently of or in association with the culture variables used to control the amount of medium perfused. The amount of carbon dioxide gas supplied may be reduced in proportion or inverse proportion to one or a combination of a plurality of these indicators. Therefore, the formulas above for the culture variables may also be used for these indicators.

In this case, the sign of the correction coefficient M is usually reversed from that used in the culture variables. For example, in a case in which the cell density, cell density increasing rate, cell count, or cell aggregate size or volume is used as an indicator, a negative M value is used because, generally, it is preferable to decrease the carbon dioxide gas concentration as these variables increase. Meanwhile, for example, in a case in which pH is used instead of the cell density as an indicator, a positive M value is used because, generally, it is preferable to decrease the carbon dioxide gas concentration as the pH decreases.

For example, the pH of the culture solution may be an indicator. In this case, the amount of carbon dioxide gas supplied can be altered (especially decreased) by changing the carbon dioxide gas concentration in the gas supplied to suppress the pH decrease. Specifically, for example, the carbon dioxide gas concentration in the gas supplied may be set to be proportional to the pH value.

The timing of starting to decrease the carbon dioxide gas concentration is arbitrary. In addition, unlike the timing of starting medium perfusion described above, the timing of starting to decrease the carbon dioxide gas concentration may be before cells form aggregates, and the decrease of the carbon dioxide gas concentration may start at the start of culture. The timing of starting to decrease the carbon dioxide gas concentration is preferably at the start of culture. As described above, in the case of starting to culture cells in the single cell state, it is not preferable to perform liquid medium perfusion before the formation of aggregates. Therefore, by adjusting the carbon dioxide gas concentration, the specific growth rate can be improved even when the culture environment cannot be controlled by perfusion.

In the suspension culture step in this perfusion mode, it is possible to isolate a part of the pluripotent stem cells during culture and to confirm the cell count or whether the undifferentiated state is maintained. For example, it is possible to confirm whether the undifferentiated state is maintained by measuring the expression of pluripotent stem cell markers expressed in pluripotent stem cells isolated during culture. For example, a pluripotent stem cell marker may include Alkaline Phosphatase, Nanog, OCT4, SOX2, TRA-1-60, c-Myc, KLF4, LIN28, SSEA-4, and SSEA-1. Examples of the method for detecting these pluripotent stem cell markers include flow cytometry, as described above.

When the positive rate for pluripotent stem cell markers in pluripotent stem cells isolated during culture is, for example, 80% or more, 90% or more, 91% or more, 92% or more, 93% or more, 94% or more, 95% or more, 96% or more, 97% or more, 98% or more, 99% or more, or 100% or less, it can be determined that the undifferentiated state is maintained. When a plurality of pluripotent stem cell markers are used, the positive rate and the undifferentiated property are determined as described above.

In addition, in this step, it is possible to confirm whether the undifferentiated state is maintained by measuring the expression of three germ layer markers (endodermal cell marker, mesodermal cell marker, and ectodermal cell marker) in the pluripotent stem cells isolated during culture. In other words, when the positive rate for all of these endodermal cell marker, mesodermal cell marker, and ectodermal cell marker are, for example, 20% or less, 10% or less, 9% or less, 8% or less, 7% or less, 6% or less, 5% or less, 4% or less, 3% or less, 2% or less, 1% or less, or the detection limit or less, it can be determined that the undifferentiated state is maintained.

An “endodermal cell marker” refers to a gene specific to endodermal cells. Examples thereof may include SOX17, FOXA2, CXCR4, AFP, GATA4, and EOMES. An endodermal cell differentiates into tissues of organs such as the digestive tract, lung, thyroid, pancreas, and liver, cells of secretory glands opening to the digestive tract, peritoneum, pleura, larynx, auditory tube, trachea, bronchi, urinary tracts (bladder most of the urethra, part of urinary duct), and the like.

A “mesodermal cell marker” refers to a gene specific to mesodermal cells. Examples thereof may include T (BRACHYURY), MESP1, MESP2, FOXF1, HAND1, EVX1, IRX3, CDX2, TBX6, MIXL1, ISL1, SNAI2, FOXC1, and PDGFRα. A mesodermal cell differentiates into body cavities and lining mesothelium, muscle, skeleton, skin dermis, and connective tissue, heart, blood vessels (including vascular endothelium), blood (including blood cells), lymph vessels, spleen, kidneys, ureters, gonads (testis, uterus, gonadal epithelium), and the like.

An “ectodermal cell marker” refers to a gene specific to ectodermal cells. Examples thereof may include FGF5, NESTIN, SOX1, and PAX6. An ectodermal cell forms the epidermis of the skin and epithelium of the terminal urethra in males, hair, nails, skin glands (including mammary and sweat glands), sensory organs (including the terminal epithelium of the oral cavity, pharynx, nose, and rectum, salivary glands), lens, peripheral nervous system, and the like. In addition, part of the ectoderm is invaginated into grooves during development to form the neural tube and also serves as the origin of neurons and melanocytes in the central nervous system, such as the brain and spinal cord.

The expression of these three germ layer markers (endodermal cell marker, mesodermal cell marker, and ectodermal cell marker) can be measured by any detection method in the art. Examples of the method for measuring the expression of three germ layer markers (endodermal cell marker, mesodermal cell marker, and ectodermal cell marker) include, but are not limited to, quantitative real-time PCR analysis, the RNA-Seq method, northern hybridization, or hybridization methods using DNA array, as well as the methods using flow cytometry described for the pluripotent stem cell markers. In quantitative real-time PCR analysis, the expression level of the marker to be measured is calculated into the relative expression level with respect to the expression level of an internal standard gene, and the expression level of the marker can be evaluated based on the relative expression level. Examples of an internal standard gene include the glyceraldehyde-3-phosphate dehydrogenase (GAPDH) gene and the R-actin (ACTB) gene. This detection method may also be used for analyzing the expression of pluripotent stem cell markers described above.

Following this step, the lower limit of specific growth rate of cells before (e.g., immediately before) the passaging step described later is preferably 0.2 day⁻¹, 0.3 day⁻¹, 0.4 day⁻¹, 0.5 day⁻¹, or 0.6 day⁻¹. In addition, the upper limit of the specific growth rate is not particularly limited. For example, it is preferably 1.5 day⁻¹, 1.4 day⁻¹, or 1.3 day⁻¹.

### 1-3-2. Passage Step

The “passage step” refers to a step of collecting cultured pluripotent stem cells or pluripotent stem cell population (e.g., cell aggregate) from a culture solution after the suspension culture step using the perfusion mode described above and subjecting the pluripotent stem cell population to dispersion treatment. “Collecting (cells or a pluripotent stem cell population)” refers to separating a culture solution and a pluripotent stem cell population to obtain the cells or the pluripotent stem cell population. A method for collecting cells or a pluripotent stem cell population may be according to a conventional method used in cell culture methods in the art, and is not particularly limited. The “dispersion treatment (of a pluripotent stem cell population)” refers to unicellularization or aggregate division of the collected pluripotent stem cell population, which may be according to a conventional method used in cell culture methods in the art.

After the suspension culture step, the cells or pluripotent stem cell population exist in a suspended state in the culture solution. Therefore, the collection thereof may be achieved by removing the liquid component of the supernatant by standing or centrifigation. They may also be collected using a filtration filter, a hollow fiber separation membrane, or the like. In the case of removing the liquid component by standing, the vessel containing the culture solution is allowed to stand for about 5 minutes, and the supernatant may be removed while leaving the sedimented cells or pluripotent stem cell population such as cell aggregates. In the case of removing the liquid component by centrifugation, the centrifugal acceleration and the treatment time may be such that the centrifugal force does not damage the cells. For example, the lower limit of the centrifugal acceleration is not particularly limited as long as cells can be sedimented, and may be, for example, 100×g, 300×g, 800×g, or 1000×g. Meanwhile, the upper limit may be any speed at which the cells are not or not easily damaged by the centrifugal force, and may be, for example, 1400×g, 1500×g, or 1600×g. The lower limit of the treatment time is not particularly limited, as long as it is the time by which the above-described centrifugal acceleration can sediment cells, and may be, for example, 30 seconds, 1 minute, 3 minutes, or 5 minutes. The upper limit thereof may be any time by which the cells are not or not easily damaged by the above-described centrifugal acceleration, and may be, for example, 10 minutes, 8 minutes, 6 minutes, or 30 seconds. In the case of removing the liquid component by filtration for collecting cell aggregates, for example, the culture solution may be passed through a nonwoven fabric or a mesh filter for removing the filtrate, thereby collecting the remaining cell aggregates. In the case of removing the liquid component by a hollow fiber separation membrane, for example, the culture solution and cells may be separated and collected using an apparatus equipped with a hollow fiber separation membrane, such as a cell concentration and washing system (KANEKA CORPORATION).

The collected cells may be washed, if necessary. The washing method is not limited. Buffer (such as PBS buffer), physiological saline, or a medium (preferably basal medium) may be used as a washing solution.

These methods for collecting and washing pluripotent stem cell populations may also be used when collecting pluripotent stem cell populations such as cell aggregates in the suspension culture step.

### (Unicellularization)

A pluripotent stem cell population collected after the suspension culture step may be “unicellularized.” Unicellularization refers to dispersing a cell population in which a plurality of cells have adhered or aggregated with each other, such as a cell aggregate, thereby achieving a free single cell state. The free single cell state may be a state in which a single cell free from a pluripotent stem cell population exist. The state is not necessarily a state in which all cells constituting a pluripotent stem cell population become free single cells.

A detachment agent and/or a chelating agent are/is used for unicellularization. Examples of a detachment agent that may be used include, but are not particularly limited to, trypsin, collagenase, pronase, hyaluronidase, elastase, and commercially available products such as Accutase (registered trademark), Accumax (registered trademark), TrypLE™ Express Enzyme (Life Technologies Japan Ltd.), TrypLE™ Select Enzyme (Life Technologies Japan Ltd.), and Dispase (registered trademark). Examples of a chelating agent that may be used include, but are not particularly limited to, EDTA and EGTA.

In the case of using trypsin for unicellularization, the lower limit of the concentration in the solution is not particularly limited as long as the pluripotent stem cell population can be dispersed, and may be, for example, 0.15% by volume, 0.18% by volume, 0.20% by volume, or 0.24% by volume. Meanwhile, the upper limit of the concentration in the solution is not particularly limited as long as the concentration does not cause an effect such as cells per se to be dissolved, and may be, for example, 0.30% by volume, 0.28% by volume, or 0.25% by volume. The treatment time depends on the concentration of trypsin, but the lower limit thereof is not particularly limited as long as the pluripotent stem cell population is well dispersed by the action of trypsin within the time. It may be, for example, 5 minutes, 8 minutes, 10 minutes, 12 minutes, or 15 minutes. Meanwhile, the upper limit of treatment time is not particularly limited as long as it does not cause an effect such as cells per se to be dissolved by the action of trypsin, and may be, for example, 30 minutes, 28 minutes, 25 minutes, 22 minutes, 20 minutes, or 18 minutes. When a commercially available detachment agent is used, it may be used at a concentration that allows the cells to be dispersed into a single state, as described in the attached protocol.

When EDTA is used for unicellularization, the lower limit of the concentration in the solution is not particularly limited as long as the pluripotent stem cell population can be dispersed, and may be, for example, 0.01 mM, 0.1 mM, or 0.5 mM. Meanwhile, the upper limit of the concentration in the solution is not particularly limited as long as the concentration does not cause an effect such as cells per se to be dissolved, and is preferably 100 mM, 50 mM, 10 mM, or 5 mM. It is preferable to use one or more of both detachment agent and chelating agent for unicellularization. After treatment with the detachment agent and/or chelating agent, unicellularization may be promoted by applying mild force to a pluripotent stem cell population such as a cell aggregate. The treatment to apply this force is not particularly limited, but for example, a method for pipetting cells together with the solution for a plurality of times, stirring with a stirring blade, or the like may be considered. Further, the cells may be passed through a strainer or mesh, if necessary.

The unicellularized cell may be collected by removing the supernatant containing the detachment agent via standing or centrifugation. The collected cell may be washed, if necessary. The conditions for centrifugation and the washing method may be the same as described above. In addition, the unicellularized cell may be subjected to the “suspension culture step after passage” described later. Until the unicellularized cell is subjected to the “suspension culture step after passage” described later, the unicellularized cell may be maintained in a state of being suspended in the washing solution, during which the temperature is not limited. For example, the temperature may be room temperature or cool refrigeration temperature. Alternatively, a cell, once unicellularized, may be frozen and preserved by a conventional method in the art, and after thawing, it may be subjected to the “suspension culture step after passage.” Further, the time for maintenance and preservation in this case is arbitrary.

### (Division of Aggregate)

In a case in which the pluripotent stem cell population collected after the suspension culture step contains a cell aggregate, “division of aggregate” may be carried out. Division of aggregate refers to dividing a cell aggregate, in which a plurality of cells have adhered or aggregated with each other, into smaller cell aggregates. The divided cell aggregate refers to a smaller cell aggregate divided from the original cell aggregate or the like and may contain cells released as single cells from the original cell aggregate or the like.

Physical and/or biochemical techniques may be used for division of aggregate. For example, although not particularly limited, the aggregate or the like may be divided by pressing the collected cell aggregate or the like through a mesh having a mesh size smaller than the size of the collected cell aggregate or by allowing hemagglutinin derived from Clostridium botulinum to act with the collected cell aggregate or the like, thereby loosening the attachment or aggregation between cells.

The divided cell aggregates may be collected by removing the supernatant by standing or centrifugation. The collected cell may be washed, if necessary. The conditions for centrifugation and the washing method may be the same as described above. Further, the divided cell aggregate may be subjected to the “suspension culture step after passage” described later.

### 1-3-3. Culture Step after Passage

The “suspension culture step after passage” that is performed after the subsequent passage step described above following the suspension culture step using the perfusion mode is a step of culturing a pluripotent stem cell population obtained after the following passage step following the suspension culture step using the perfusion mode. In this step, a cell may remain undifferentiated to grow, or a cell may be induced to differentiate without being retained in an undifferentiated state. The culture method after the passage step is not particularly limited, but the passage is preferably passage from suspension culture to suspension culture. Performing the suspension culture of pluripotent stem cells in the perfusion mode with an appropriate amount of medium perfused in the suspension culture step described above makes it possible to maintain a high cell count maintaining rate at the start of this step and improve subsequent production efficiency.

In principle, the cell culture method in this step is in accordance with the one described in “1-3-1. Suspension Culture Step” using the perfusion mode described above. Therefore, the descriptions common to the above-described method in the suspension culture step using the perfusion mode is omitted, and only the characteristic points of this step are described in detail.

### (Cells)

Cells used in this step are cells prepared in the passage step. The cell type is pluripotent stem cell as described in the suspension culture step using the perfusion mode, and for example, pluripotent stem cells such as iPS cells and ES cells may be preferably used. In addition, the state of the cells when seeded in the medium is preferably a single cell state.

### (Cell Container)

The cell vessel used in this step may be the same as described for the suspension culture step using the perfusion mode.

### (Medium)

In this step, the perfusion mode is not necessarily employed, and the batch mode or other mode may be used. In a case in which the passage step is further performed after this step as well to perform passage to suspension culture, it is preferable to perform suspension culture by the perfusion mode in this step as well.

The type of medium may be a medium capable of proliferating and/or maintaining undifferentiated cells as described in the suspension culture step using the perfusion mode or a medium containing certain additives to differentiate cells without maintaining them.

### (Seeding Density)

When the cells prepared in the above-described passage step are seeded in a new medium, the cell density (seeding density) is not particularly limited and may be adjusted accordingly in consideration of the culture time, cell state after culture, and the cell count required after culture. Although not limited, in general, the lower limit thereof may be in a range of 0.01×10⁵ cells/mL or more, 0.1×10⁵ cells/mL or more, 1×10⁵ cells/mL or more, or 2×10⁵ cells/mL or more, and the upper limit thereof may be in a range of 20×10⁵ cells/mL or less or 10×10⁵ cells/mL or less. Specifically, for example, the lower limit of the seeding density may be 2×10⁵ cells/mL or more, and the upper limit thereof may be 4×10⁵ cells/mL or less.

In general, it is known that a certain number of cells passaged from suspension culture die after seeding, resulting in a temporal decrease in the cell density compared to the seeding density. However, according to the present invention, the lower limit of the proportion of the cell density on day 1 after seeding is preferably 60%, 70%, 80%, or 90% with respect to the seeding density.

### (Culture Method)

In this step, the fluid state of the medium during culture is arbitrary. In other words, the culture may be static culture or flow culture described in the suspension culture step using the perfusion mode. However, typically, flow culture is employed for suspension culture. Meanwhile, static culture may be preferable when cells are induced to differentiate. In the case of adopting static culture for suspension culture, although not limited, a suspension culture plate or multi-well plate treated to prevent cell attachment may be used.

### 1-3-4. Treatment Following Culture Step after Passage

After the culture step after passage described above, a step of collecting a cell or a pluripotent stem cell population may be performed. This step is optional. In the step of collecting a cell or a pluripotent stem cell population, the cell or pluripotent stem cell population is separated from the culture solution by a conventional method, and the separated cell or pluripotent stem cell population is collected. In this connection, a cell may be collected as a single cell by detachment or dispersion treatment from an adjacent pluripotent stem cell or may be collected as a pluripotent stem cell population. A specific method is described in detail in “1-3-2. Passage Step” above. The collected cell or cell aggregate may be directly or, if necessary, washed with buffer (such as PBS buffer), physiological saline, or a medium (preferably a medium used in the suspension culture step after the passage step or a basal medium) and then subjected to a desired step.

### 1-4. Effects

According to the method for producing a pluripotent stem cell population of the present invention, pluripotent stem cells can be subjected to suspension culture in an excellent state using the perfusion mode with an appropriate amount of medium perfused, following which the death of pluripotent stem cells can be suppressed when the pluripotent stem cells are passaged from the suspension culture. Suppressing the pluripotent stem cell death is synonymous with maintaining a high cell count when performing suspension culture of pluripotent stem cells passaged from suspension culture. In other words, comparing the cell count when performing suspension culture of pluripotent stem cells using the batch process or the perfusion mode with an inappropriate amount of medium perfused and then passaging the cells to suspension culture, and the cell count when performing suspension culture of pluripotent stem cells using the perfusion mode with an appropriate amount of medium perfused and then passaging the cells to suspension culture under arbitrary conditions, the latter is higher.

In addition, culture efficiency can be improved by using perfusion culture in which the amount of medium perfused is controlled in line with the progress of culture in accordance with the characteristics of a pluripotent stem cell. Improving the culture efficiency of a pluripotent stem cell is synonymous with suppressing the decrease in the growth ability and growth rate of a cell during suspension culture and/or improving the growth ability and growth rate of a cell during suspension culture. In other words, comparing the cell count in the culture solution cultured by the conventional method and the cell count in the culture solution cultured for the same period by the method of the present invention, the latter is higher.

Further, the culture efficiency can be remarkably improved by altering the amount of carbon dioxide gas supplied in line with the progress of the suspension culture in accordance with the characteristics of the pluripotent stem cell. In addition, the culture efficiency can be synergistically improved by using perfusion culture, in which the amount of medium perfused is controlled in accordance with the progress of the culture, in combination. In other words, comparing the cell count in the culture solution cultured by the conventional method and the cell count in the culture solution cultured for the same period by the method of the present invention, the latter is higher.

The cell death in suspension culture can be suppressed, and the production efficiency can be improved by applying the method for producing a pluripotent stem cell population of the present invention. Therefore, a pluripotent stem cell population can be produced efficiently by suspension culture. In other words, a pluripotent stem cell population consisting of a desired number of cells can be produced in a relatively short period of time. Accordingly, the cost associated with the pluripotent stem cell population production can be greatly reduced.

### EXAMPLES

Hereinafter, the method for producing a pluripotent stem cell population according to the present invention will be described in more detail with reference to Examples. However, the scope of the present invention is not intended to be limited by the following Examples.

### Embodiment A: Production of Cell Population Via Passage after Suspension Culture

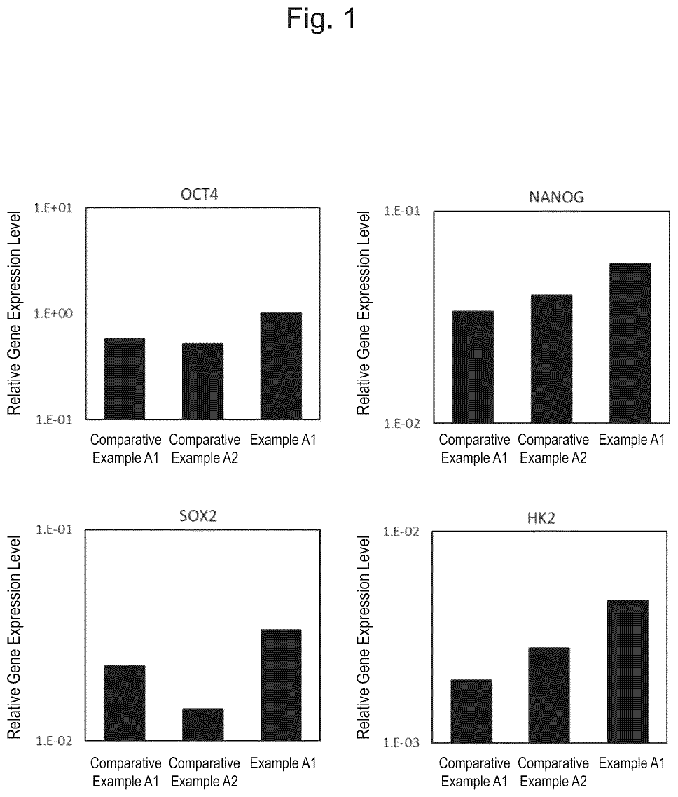

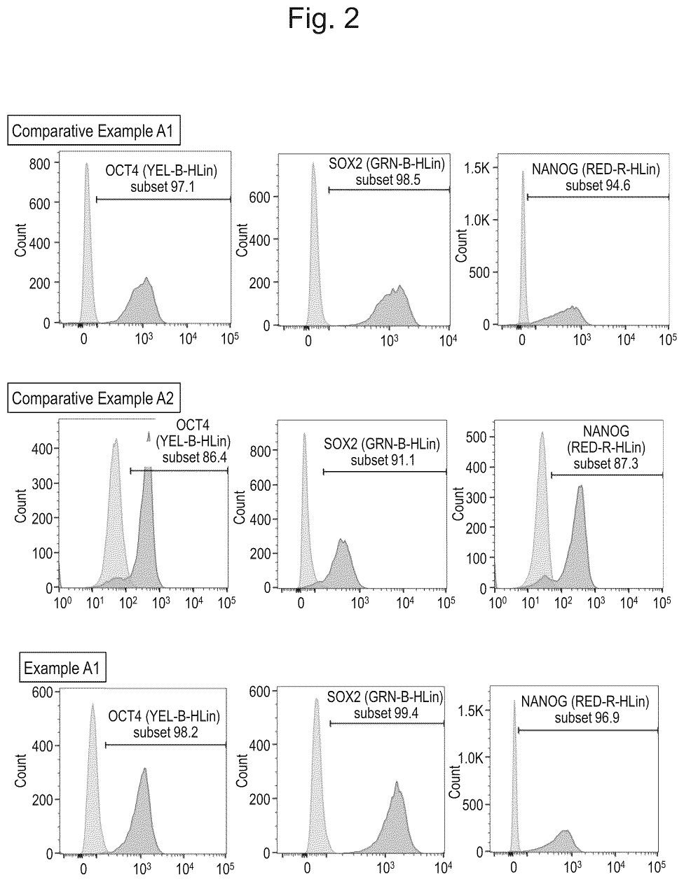

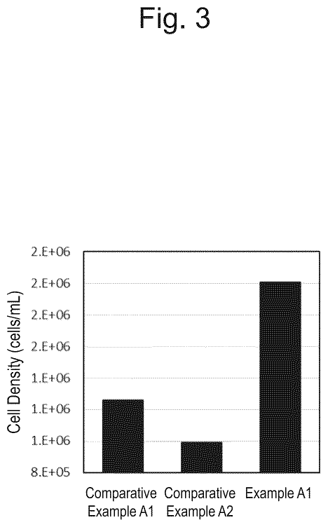

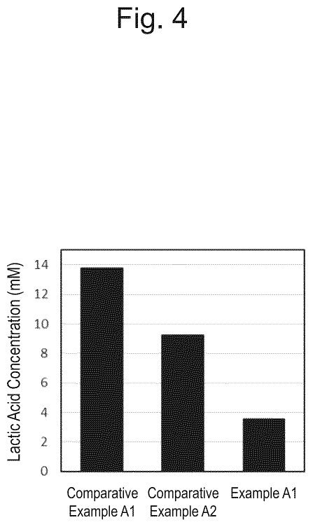

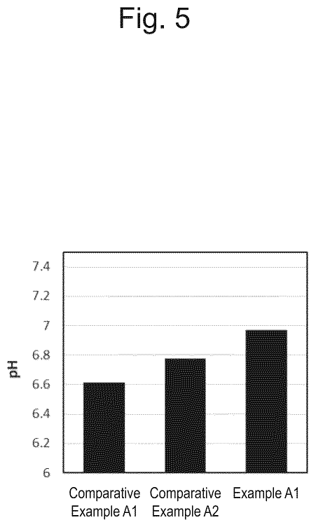

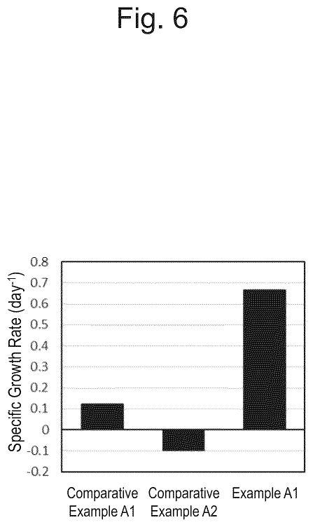

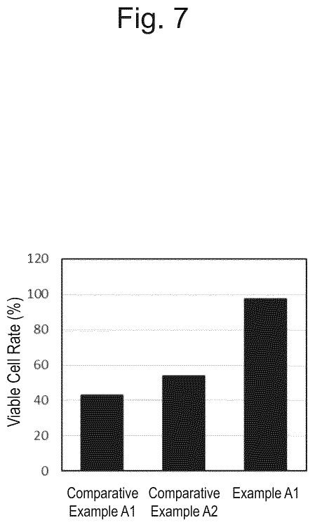

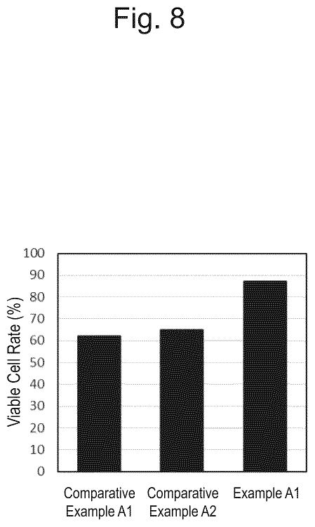

The present embodiment corresponds to the Reference Examples, Comparative Examples, Examples, and Evaluation Examples in Japanese Patent Application No. 2021-051004, on which the priority of the present application is based. To distinguish the Examples and the like from those in the other embodiments, the Examples and the like in the present embodiment are expressed as the Examples and the like in Japanese Patent Application No. 2021-051004 plus “A.” Each of Figures of the present application and Figures in Japanese Patent Application No. 2021-051004 correspond to as follows: FIG. 1 and FIG. 1, FIG. 2 and FIG. 2, FIG. 3 and FIG. 3, FIG. 4 and FIG. 4, and FIG. 5 and FIG. 5, FIG. 6 and FIG. 6, FIG. 7 and FIG. 7, and FIG. 8 and FIG. 8. In addition, each of Tables of the present application and Tables in Japanese Patent Application No. 2021-051004 correspond to as follows: Table 1 and Table 1, Table 2 and Table 2, Table 3 and Table 3, Table 4 and Table 4, Table 5 and Table 5, Table 6 and Table 6, Table 7 and Table 7, Table 8 and Table 8, Table 9 and Table 9, Table 10 and Table 10, and Table 11 and Table 11. Further, each of SEQ ID NOs of the present application and SEQ ID NOs in Japanese Patent Application No. 2021-051004 correspond to as follows: SEQ ID NO 1 and SEQ ID NO 1, SEQ ID NO 2 and SEQ ID NO 2, SEQ ID NO 3 and SEQ ID NO 3, SEQ ID NO 4 and SEQ ID NO 4, SEQ ID NO 5 and SEQ ID NO 5, SEQ ID NO 6 and SEQ ID NO 6, SEQ ID NO 7 and SEQ ID NO 7, SEQ ID NO 8 and SEQ ID NO 8, SEQ ID NO 9 and SEQ ID NO 9, and SEQ ID NO 10 and SEQ ID NO 10.

### Reference Example A1: Adherent Culture of Human iPS Cell Line 1383D6

A human iPS cell line 1383D6 (Center for iPS Cell Research and Application, Kyoto University) was seeded at 10000 cells/cm² on a cell culture dish coated with Vitronectin (VTN-N) Recombinant Human Protein, Truncated (Thermo Fisher Scientific Inc.) at 0.5 μg/cm², followed by adherent culture at 37° C. in a 5% CO₂ atmosphere. StemFit (registered trademark) AK02N (AJINOMOTO CO., INC.) was used as a medium, and medium change was performed every day. Y-27632 (FUJIFILM Wako Pure Chemical Corporation) was added to the medium at a final concentration of 10 μM only at the time of cell seeding. The day of cell seeding was set as day 0 of culture, and cells were detached from the culture surface by treatment with Accutase (Innovative Cell Technologies, Inc.) for 5 minutes and dispersed as single cells by pipetting on day 4 of culture for passage. The cells were suspended in StemFit (registered trademark) AK02N (AJINOMOTO CO., INC.) containing Y-27632 at a final concentration of 10 μM, and some of the cells were stained with trypan blue to measure the viable cell count. Then, cell seeding was performed to continue adherent culture in the same manner.

### Example A1: Suspension Culture of Human iPS Cell Line 1383D6 in Perfusion Mode

The cells cultured in Reference Example A1 were seeded for suspension culture at the time of passage. BioBlu 0.3c Single-Use Vessel (Eppendorf SE) was used as a culture vessel. DASbox (Eppendorf SE) was used as a reactor system for controlling culture. A pH sensor and a medium perfusion pump provided with DASbox were calibrated in advance according to the method specified by the manufacturer. The cells were seeded such that the amount of culture solution was 142 mL and the cell density at the start of culture was 3.0×10⁵ cells/mL, and then the culture was started. StemFit (registered trademark) AK02N (AJINOMOTO CO., INC.) supplemented with 10 μM Y-27632 and 20 μM IWR-1 endo was used as a medium. During the culture, the culture temperature was maintained at 37° C., the carbon dioxide gas concentration in the aeration gas was maintained at 5%, the oxygen concentration was maintained at about 20%, and the aeration gas rate was maintained at 3.0 L/h to perform top surface aeration of the culture solution. The stirring speed was set to 130 rpm until day 1 of culture and 120 rpm afterward. The start of culture was set at day 0 of culture, and medium perfusion was started at day 1 of culture. The amount of medium perfused was changed at 1-hour intervals, as shown in Table 1. The amount of medium perfused per unit time at the start of perfusion (reference perfusion rate) was determined to be F₀=7.40 mL/h obtained by multiplying the result of dividing a culture volume of 142 mL with 24 hours, 5.92 mL, by a constant of 1.25 set based on a cell density of 80% at the start of perfusion with respect to an assumed seeding concentration of 3.0×10⁵ cells/mL. Until day 3 of culture, the amount of medium perfused per unit time was set using Formula 2 above setting C₀ as the cell count calculated from a cell density of 80% at the start of perfusion with respect to an assumed seeding concentration of 3.0×10⁵ cells/mL. C was the number of cells calculated from the empirically predicted transition of the assumed cell density. A constant M for correcting the influence of cell lines and the like was set to 1. In addition, after day 3 of culture, Formula 3 above was used to set the amount of medium perfused per unit time by calculating K with the amount of lactic acid produced by metabolism per cell per unit time which is assumed from the information in advance as L₀, and the amount of lactic acid produced by metabolism per cell per unit time in each culture time as L on the cell line on day 3 of culture. A constant M for correcting the influence of cell lines and the like was set to 1. The composition of the medium used for perfusion was switched between days 1 to 2 of culture and days 2 to 4 of culture such that the concentration of Y-27632 was different, 4.7 μM for day 1 to day 2 of culture and 2 μM for day 2 to day 4 of culture. StemFit (registered trademark) AK02N (AJINOMOTO CO., INC.) supplemented with 20 μM IWR-1 endo and 1 μM LY333531 was used for both media. To remove the medium and not the cell aggregate by suctioning from the culture solution, the medium was removed by passing through a sintered wire mesh filter having an opening of 30 μm.

TABLE 1

| Elapsed culture time | Flow rate |
|---|---|
| (d. h:m:s) | (mL/h) |
| 1.00:00:00 | 7.40 |
| 1.01:00:00 | 7.65 |
| 1.02:00:00 | 7.91 |
| 1.03:00:00 | 8.18 |
| 1.04:00:00 | 8.46 |
| 1.05:00:00 | 8.74 |
| 1.06:00:00 | 9.04 |
| 1.07:00:00 | 9.34 |
| 1.08:00:00 | 9.66 |
| 1.09:00:00 | 9.99 |
| 1.10:00:00 | 10.33 |
| 1.11:00:00 | 10.68 |
| 1.12:00:00 | 11.04 |
| 1.13:00:00 | 11.41 |
| 1.14:00:00 | 11.80 |
| 1.15:00:00 | 12.20 |
| 1.16:00:00 | 12.61 |
| 1.17:00:00 | 13.04 |
| 1.18:00:00 | 13.48 |
| 1.19:00:00 | 13.94 |
| 1.20:00:00 | 14.41 |
| 1.21:00:00 | 14.90 |
| 1.22:00:00 | 15.41 |
| 1.23:00:00 | 15.93 |
| 2.00:00:00 | 16.47 |
| 2.01:00:00 | 17.03 |
| 2.02:00:00 | 17.60 |
| 2.03:00:00 | 18.20 |
| 2.04:00:00 | 18.82 |
| 2.05:00:00 | 19.46 |
| 2.06:00:00 | 20.12 |
| 2.07:00:00 | 20.80 |
| 2.08:00:00 | 21.50 |
| 2.09:00:00 | 22.23 |
| 2.10:00:00 | 22.98 |
| 2.11:00:00 | 23.76 |
| 2.12:00:00 | 24.57 |
| 2.13:00:00 | 25.40 |
| 2.14:00:00 | 26.26 |
| 2.15:00:00 | 27.15 |
| 2.16:00:00 | 28.07 |
| 2.17:00:00 | 29.02 |
| 2.18:00:00 | 30.01 |
| 2.19:00:00 | 31.03 |
| 2.20:00:00 | 32.08 |
| 2.21:00:00 | 33.16 |
| 2.22:00:00 | 34.29 |
| 2.23:00:00 | 35.45 |
| 3.00:00:00 | 36.65 |
| 3.01:00:00 | 37.64 |
| 3.02:00:00 | 38.66 |
| 3.03:00:00 | 39.70 |
| 3.04:00:00 | 40.76 |
| 3.05:00:00 | 41.85 |
| 3.06:00:00 | 42.97 |
| 3.07:00:00 | 44.12 |
| 3.08:00:00 | 45.30 |
| 3.09:00:00 | 46.50 |
| 3.10:00:00 | 47.73 |
| 3.11:00:00 | 49.00 |
| 3.12:00:00 | 50.29 |
| 3.13:00:00 | 51.62 |
| 3.14:00:00 | 52.98 |
| 3.15:00:00 | 54.37 |
| 3.16:00:00 | 55.80 |
| 3.17:00:00 | 57.26 |
| 3.18:00:00 | 58.75 |
| 3.19:00:00 | 60.28 |
| 3.20:00:00 | 61.85 |
| 3.21:00:00 | 63.45 |
| 3.22:00:00 | 65.09 |
| 3.23:00:00 | 66.77 |

### Comparative Example A1: Suspension Culture of Human iPS Cell Line 1383D6 in Batch Mode

Cell culture was performed under the same conditions as in Example A1, except that medium change was perform by the batch mode. The culture medium was changed in its entirety every day. StemFit (registered trademark) AK02N (AJINOMOTO CO., INC.) was used as a medium. Table 2 lists the concentrations of additives in the medium used in each medium change.

TABLE 2

| | Y-27632 | IWR-1 endo | LY333531 |
|---|---|---|---|
| | (μM) | (μM) | (μM) |
| Day 0 to day 1 of culture | 10 | 20 | 0 |
| Day 1 to day 2 of culture | 6.9 | 20 | 0.58 |
| Day 2 to day 3 of culture | 2.7 | 20 | 0.99 |
| Day 3 to day 4 of culture | 2 | 20 | 1 |

### Comparative Example A2: Suspension Culture of Human iPS Cell Line 1383D6 in Constant Flow Rate Perfusion Mode

Cell culture was performed under the same conditions as in Example A1, except for the amount of medium perfused. The medium perfusion was started on day 1 of culture as in Example A1. The culture was performed at a constant amount of medium perfused of 5.92 mL/h such that the amount of medium used per day was equal to that in Comparative Example A1.

### Evaluation Example A1: Quantitative Real-Time PCR Analysis

Quantitative real-time PCR analysis was performed by the following procedure. The cells on day 4 of culture in Comparative Examples A1 and A2 and Example A1 were dissolved using TRIzol™ Reagent (Thermo Fisher Scientific Inc.). Total RNA was isolated and purified using PureLink (registered trademark) RNA Mini Kit (Thermo Fisher Scientific Inc.) from the solution of the cells dissolved with TRIzol™ Reagent. The concentration of purified RNA was measured using BioSpec-nano (SHIMADZU CORPORATION), thereby fractionating 500 ng of purified RNA. 10 μL solution is prepared with the adding 2 μL of ReverTra Ace (registered trademark) qPCR RT Master mix (TOYOBO CO., LTD.) and Rnase Free dH₂O to the fractionated RNA. cDNA synthesis was performed using SimpliAmp Thermal Cycler (Thermo Fisher Scientific Inc.). Reaction conditions for cDNA synthesis was as follows: a reaction at 37° C. was performed for 15 minutes, and then a reaction at 50° C. for 5 minutes and a reaction at 98° C. for 5 minutes were performed sequentially, followed by cooling to 4° C. The synthesized cDNA solution was diluted 100-fold with 10 mM Tris-HCl (pH 8.0; NACALAI TESQUE, INC.) and added at 5 μL/well to a 384-well PCR plate (Thermo Fisher Scientific Inc.). KOD SYBR (registered trademark) qPCR Mix (TOYOBO CO., LTD.), a forward primer prepared to be 50 μM, a reverse primer prepared to be 50 μM, and DEPC-treated water (NACALAI TESQUE, INC.) were mixed at a ratio of 100:1:1:48. This mixed solution was added at 15 μl/well to the 384-well PCR plate and mixed. ACTB, OCT4, SOX2, NANOG, and HK2 were used as primers. The 384-well PCR plate was centrifuged to remove air bubbles in the wells, and quantitative real-time PCR analysis was performed using QuantStudio 7 Flex Real-Time PCR System (Thermo Fisher Scientific Inc.). Table 3 lists the reaction conditions.

TABLE 3

| Step | Temperature | Time | | Number of cycles |
|---|---|---|---|---|
| 1 Initial denaturation | 98° C. | 1 | min | — |
| 2 Denaturation | 98° C. | 15 | sec | 5 cycles |
| 3 Annealing, Elongation | 68° C. | 30 | sec | |
| 4 Denaturation | 98° C. | 15 | sec | 40 cycles |
| 5 Annealing | 60° C. | 10 | sec | |
| 6 Elongation | 68° C. | 30 | sec | |
| 7 Melting curve | 95° C. | 15 | sec | — |
| | 60° C. | 1 | min | |
| | 98° C. | 15 | sec | |

The base sequences of the primers used for quantitative real-time PCR analysis are shown below.

ACTB (Forward):
(SEQ ID NO: 1)
5′-CCTCATGAAGATCCTCACCGA-3′
ACTB (Reverse):
(SEQ ID NO: 2)
5′-TTGCCAATGGTGATGACCTGG-3′
OCT4 (Forward):
(SEQ ID NO: 3)
5′-AGTGGGTGGAGGAAGCTGACAAC-3′
OCT4 (Reverse):
(SEQ ID NO: 4)
5′-TCGTTGTGCATAGTCGCTGCTTGA-3′
SOX2 (Forward):
(SEQ ID NO: 5)
5′-CACCAATCCCATCCACACTCAC-3′
SOX2 (Reverse):
(SEQ ID NO: 6)
5′-GCAAAGCTCCTACCGTACCAC-3′
NANOG (Forward):
(SEQ ID NO: 7)
5′-AGCCTCCAGCAGATGCAAGAACTC-3′
NANOG (Reverse):
(SEQ ID NO: 8)
5′-TTGCTCCACATTGGAAGGTTCCCA-3′
HK2 (Forward):
(SEQ ID NO: 9)
5′-ACCCAGTTACACACTTACCATC-3′
HK2 (Reverse):
(SEQ ID NO: 10)
5′-TGCAGTGAGCTATGATCATACC-3′

Table 4 and FIG. 1 show the results of gene expression measurement.

TABLE 4
Relative gene expression level to ACTB (2^{-ΔCt})

| | Comparative | Comparative | |
|---|---|---|---|
| | Example A1 | Example A2 | Example A1 |
| OCT4 | 5.83 × 10⁻¹ | 5.15 × 10⁻¹ | 1.02 |
| NANOG | 3.37 × 10⁻² | 3.99 × 10⁻² | 5.67 × 10⁻² |
| SOX2 | 2.26 × 10⁻² | 1.41 × 10⁻² | 3.34 × 10⁻² |
| HK2 | 2.00 × 10⁻³ | 2.82 × 10⁻³ | 4.74 × 10⁻³ |

As shown in Table 4 and FIG. 1, the cells obtained in Comparative Examples A1 and A2 and Example A1 were found to have maintained the undifferentiated property in pluripotent stem cells. The expression levels of the undifferentiation markers and the HK2 gene involved in metabolism tended to be increased in Example A1.

### Evaluation Example A2: Flow Cytometry Analysis

The cell aggregates on day 4 of culture in Comparative Examples A1 and A2 and Example A1 were treated with Accutase and dispersed as single cells by pipetting. The cells were washed with PBS (phosphate-buffered saline). Thereafter, fixation, permeabilization, and blocking were performed using eBioscience Foxp3 Transcription Factor Staining Buffer Set (Thermo Fisher Scientific Inc.). Then, the cell sample was divided into four, and each of them was resuspended to 50 μL using a buffer provided with the eBioscience Foxp3 Transcription Factor Staining Buffer Set (Thermo Fisher Scientific Inc.). Fluorescently-labeled anti-OCT4, anti-SOX2, and anti-NANOG antibodies were added to and mixed with one of the samples, and FMO controls were prepared by mixing each of three samples with antibodies in which each antibody was removed from the three fluorescently-labeled antibodies. Staining was performed at 4° C. for 1 hour in a light-proof state. Table 5 lists the antibodies used and their amounts added.

TABLE 5

| | Manufacturer, Model number | Amount added | |
|---|---|---|---|
| Fluorescently-labeled | Becton, Dickinson and | 10 | μL |
| anti-OCT4 antibody | Company, 560186 | | |
| Fluorescently-labeled | BioLegend, 656110 | 1 | μL |
| anti-SOX2 antibody | | | |
| Fluorescently-labeled | Becton, Dickinson and | 2.5 | μL |

| | |
|---|---|
| anti-NANOG antibody | Company, 561300 |

After washing once with 3% FBS (fetal bovine serum)/PBS, the cells passed through a cell strainer were analyzed using Guava easyCyte 8HT (Luminex Corporation). For the FMO control samples, all regions where the cell population with the strong fluorescence intensity was 1.0% or less were selected in the cell populations extracted by the FSC/SSC dot plot. For samples treated with the anti-OCT4, anti-SOX2, and anti-NANOG antibodies, the proportion of cells contained within the regions was calculated in the cell populations extracted from the FSC/SSC dot plot. This was defined as the proportion of cells positive for OCT4, SOX2, and NANOG. Table 6 and FIG. 2 show the results.

TABLE 6

| | Proportion of | Proportion of | Proportion of |
|---|---|---|---|
| | cells positive | cells positive | cells positive |
| | for OCT4 (%) | for SOX2 (%) | for NANOG (%) |
| Comparative | 97.1 | 98.5 | 94.6 |
| Example A1 | | | |
| Comparative | 86.4 | 87.3 | 91.1 |
| Example A2 | | | |
| Example A1 | 98.2 | 99.4 | 96.9 |

As shown in Table 6 and FIG. 2, the proportion of cells exhibiting positive for OCT4, SOX2, and NANOG, which are undifferentiation markers, was 90% or more in both the cells obtained in Comparative Example A1 and in Example A1. Meanwhile, in Comparative Example A2, the positive rate for OCT4 and NANOG were slightly below 90%, indicating that the cell population deviating from the undifferentiated state was slightly emerged, suggesting that perfusion with a constant flow rate may not allow repeated passages due to the deviation from the undifferentiated state.

### Evaluation Example A3: Confirmation of Cell Density at Passage

The cell aggregates on day 4 of culture in Comparative Examples A1 and A2 and Example A1 were treated with Accutase (Innovative Cell Technologies, Inc.) for 10 minutes, and unicellularized by pipetting. The cells were suspended in StemFit (registered trademark) AK02N (AJINOMOTO CO., INC.) containing Y-27632 at a final concentration of 10 μM, and some of the cells were stained with trypan blue to measure the viable cell count. Table 7 and FIG. 3 show the results.

TABLE 7

| | Viable cell density at passage/ |
|---|---|
| | on day 4 of culture (cells/mL) |
| Comparative Example A1 | 1.26 × 10⁶ |
| Comparative Example A2 | 0.99 × 10⁶ |
| Example A1 | 2.00 × 10⁶ |

As shown in Table 7 and FIG. 3, the cell density of Example A1 was the highest, indicating higher production efficiency.

### Evaluation Example A4: Measurement of Lactic Acid Concentration in Culture Solution at Passage

The culture solutions on day 4 of culture in Comparative Examples A1 and A2 and Example A1 were collected for measuring the lactic acid concentration using Multiple Functions Biosensor BF-7D (Oji Scientific Instruments). Table 8 and FIG. 4 show the results.

TABLE 8

| | Lactic acid concentration at |
|---|---|
| | passage/on day 4 of culture (mM) |
| Comparative Example A1 | 13.76 |
| Comparative Example A2 | 9.2 |
| Example A1 | 3.52 |

As shown in Table 8 and FIG. 4, the lactic acid concentration was kept low in Example A1, indicating that a more suitable environment for pluripotent stem cells could be maintained.

### Evaluation Example A5: Measurement of pH in Culture Solution at Passage

The pH of the culture solutions on day 4 of culture in Comparative Examples A1 and A2 and Example A1 were measured using a pH sensor provided with the reactor system DASbox. Table 9 and FIG. 5 show the results.

TABLE 9

| | pH in medium at passage/ |
|---|---|
| | on day 4 of culture |
| Comparative Example A1 | 6.61 |
| Comparative Example A2 | 6.68 |
| Example A1 | 6.97 |

As shown in Table 9 and FIG. 5, the pH range was closer to that suitable for pluripotent stem cells in Example A1, indicating that a suitable environment could be maintained.

### Evaluation Example A6: Confirmation of Specific Growth Rate at Passage

The cell aggregates on day 3 of culture in Comparative Examples A1 and A2 and Example A1 were treated with Accutase (Innovative Cell Technologies, Inc.) for 10 minutes, and unicellularized by pipetting. The cells were suspended in StemFit (registered trademark) AK02N (AJINOMOTO CO., INC.) containing Y-27632 at a final concentration of 10 μM, and some of the cells were stained with trypan blue to measure the viable cell count. Table 10 and FIG. 6 show the results of calculating the specific growth rate 24 hours before passage from the measurement results and the results of Evaluation Example A3.

TABLE 10

| | Specific growth rate 24 |
|---|---|
| | hours before passage (day⁻¹) |
| Comparative Example A1 | 0.12 |
| Comparative Example A2 | −0.097 |
| Example A1 | 0.67 |

As shown in Table 10 and FIG. 6, the specific growth rate of Example A1 was the highest, indicating that the state of cells at the time of passage was more suitable.

### Example A2: Passage from Suspension Culture to Suspension Culture of Human iPS Cell Line 1383D6

The cell aggregates on day 4 of culture in Comparative Examples A1 and A2 and Example A1 were treated with Accutase (Innovative Cell Technologies, Inc.) for 10 minutes, and unicellularized by pipetting. The cells were suspended in StemFit (registered trademark) AK02N (AJINOMOTO CO., INC.) containing Y-27632 at a final concentration of 10 μM and IWR-1 endo at a final concentration of 20 μM, and some of the cells were stained with trypan blue to measure the viable cell count. Cell suspensions were prepared using StemFit (registered trademark) AK02N (AJINOMOTO CO., INC.) containing Y-27632 at a final concentration of 10 μM and IWR-1 endo at a final concentration of 20 μM such that each cell suspension included 2×10⁵ cells per 1 mL. Each cell suspension was seeded in an amount of 30 mL in a 30 mL reactor (ABLE Corporation). A stirring blade was rotated in the reactor, in which the cells were seeded, at a speed of 135 rpm on a magnetic stirrer for performing suspension culture at 37° C. in a 5% CO₂ environment.

### Evaluation Example A7: Confirmation of Efficiency of Passage from Suspension Culture to Suspension Culture

The cell aggregates on day 1 of culture in each reactor of Example A2 obtained by performing passage of cells on day 4 of culture in Comparative Examples A1 and A2 and Example A1 from suspension culture to suspension culture were treated with Accutase (Innovative Cell Technologies, Inc.) for 10 minutes, and unicellularized by pipetting. The cells were suspended in StemFit (registered trademark) AK02N (AJINOMOTO CO., INC.) containing Y-27632 at a final concentration of 10 μM, and some of the cells were stained with trypan blue to measure the viable cell count. Table 11 and FIGS. 7 and 8 show the results.

TABLE 11

| | Cell density maintaining | Viable cell |
|---|---|---|
| | rate for 24 hours after | rate 24 hours after |
| | passage (%) | passage (%) |
| Comparative Example A1 | 43 | 62 |
| Comparative Example A2 | 54 | 65 |
| Example A1 | 97 | 87 |

As shown in Table 11 and FIGS. 7 and 8, it was confirmed that when cells from Example A1 were passaged from suspension culture to suspension culture in Example A2, the passage efficiency was remarkably improved. Combined with the cell density reached at the time of passage shown in Evaluation Example A3, the results indicate that remarkably excellent productivity in suspension culture was demonstrated in Example A1.

### Embodiment B: Production of Cell Population Using Strict Control of Amount of Medium Perfused

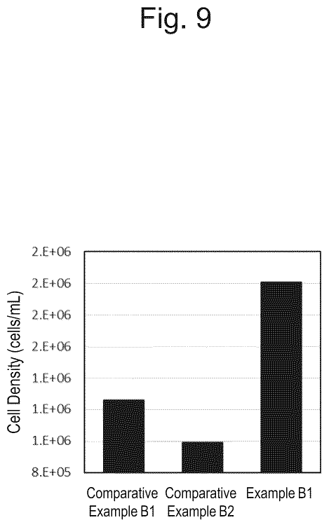

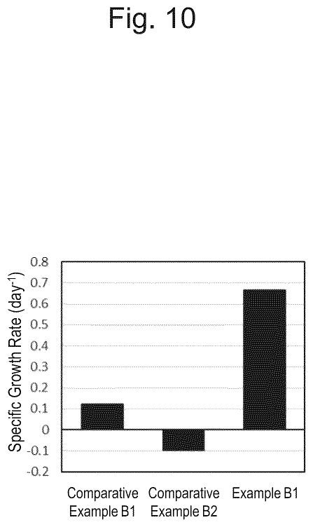

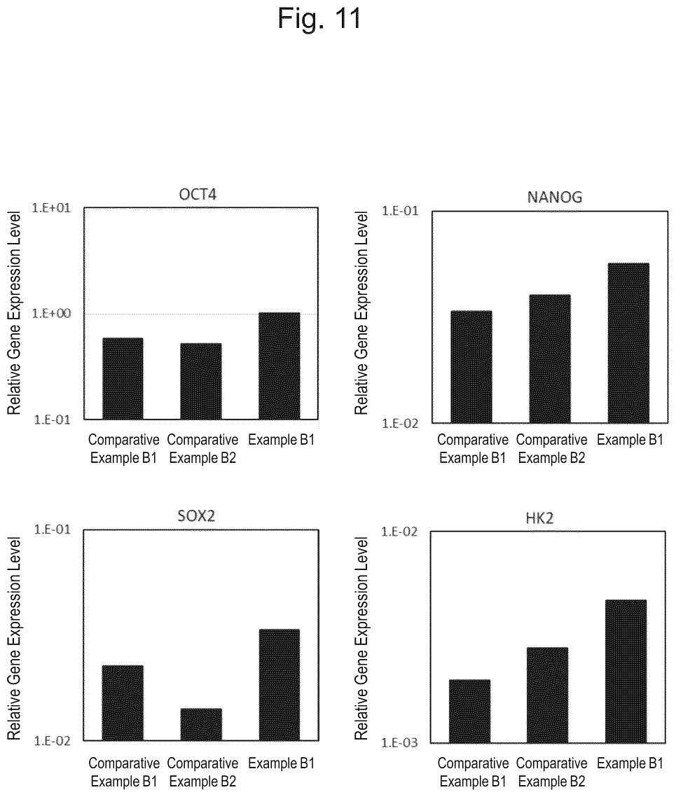

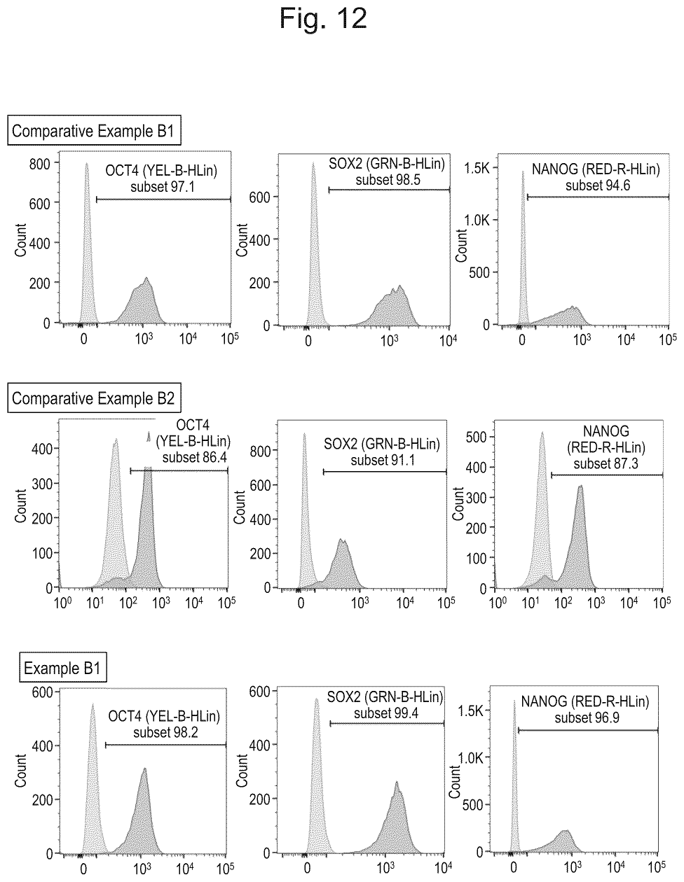

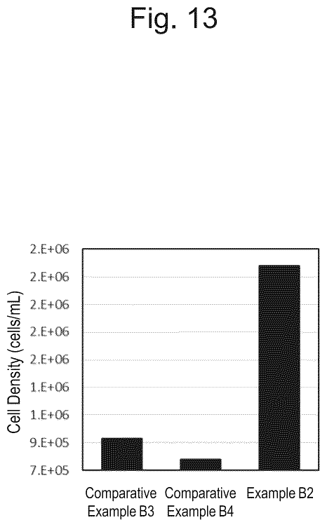

The present embodiment corresponds to the Reference Examples, Comparative Examples, Examples, and Evaluation Examples in Japanese Patent Application No. 2021-051006, on which the priority of the present application is based. To distinguish the Examples and the like from those in the other embodiments, the Examples and the like in the present embodiment are expressed as the Examples and the like in Japanese Patent Application No. 2021-051006 plus “B.” Each of Figures of the present application and Figures in Japanese Patent Application No. 2021-051006 correspond to as follows: FIG. 9 and FIG. 1, FIG. 10 and FIG. 2, FIG. 11 and FIG. 3, FIG. 12 and FIG. 4, and FIG. 13 and FIG. 5. In addition, each of Tables of the present application and Tables in Japanese Patent Application No. 2021-051006 correspond to as follows: Table 12 and Table 1, Table 13 and Table 2, Table 14 and Table 3, Table 15 and Table 4, Table 16 and Table 5, Table 17 and Table 6, Table 18 and Table 7, Table 19 and Table 8, Table 20 and Table 9, Table 21 and Table 10, and Table 22 and Table 11. Further, each of SEQ ID NOs of the present application and SEQ ID NOs in Japanese Patent Application No. 2021-051006 correspond to as follows: SEQ ID NO 11 and SEQ ID NO 1, SEQ ID NO 12 and SEQ ID NO 2, SEQ ID NO 13 and SEQ ID NO 3, SEQ ID NO 14 and SEQ ID NO 4, SEQ ID NO 15 and SEQ ID NO 5, SEQ ID NO 16 and SEQ ID NO 6, SEQ ID NO 17 and SEQ ID NO 7, and SEQ ID NO 18 and SEQ ID NO 8, SEQ ID NO 19 and SEQ ID NO 9, and SEQ ID NO 20 and SEQ ID NO 10.

### Reference Example B1: Adherent Culture of Human iPS Cell Line 1383D6

A human iPS cell line 1383D6 (Center for iPS Cell Research and Application, Kyoto University) was seeded at 10000 cells/cm² on a cell culture dish coated with Vitronectin (VTN-N) Recombinant Human Protein, Truncated (Thermo Fisher Scientific Inc.) at 0.5 μg/cm², followed by adherent culture at 37° C. in a 5% CO₂ atmosphere. StemFit (registered trademark) AK02N (AJINOMOTO CO., INC.) was used as a medium, and medium change was performed every day. Y-27632 (FUJIFILM Wako Pure Chemical Corporation) was added to the medium at a final concentration of 10 μM only at the time of cell seeding. The day of cell seeding was set as day 0 of culture, and cells were detached from the culture surface by treatment with Accutase (Innovative Cell Technologies, Inc.) for 5 minutes and dispersed as single cells by pipetting on day 4 of culture for passage. The cells were suspended in StemFit (registered trademark) AK02N containing Y-27632 at a final concentration of 10 μM, and some of the cells were stained with trypan blue to measure the viable cell count. Then, cell seeding was performed to continue adherent culture in the same manner.

### Example B1: Suspension Culture of Human iPS Cell Line 1383D6 in Perfusion Mode

The cells cultured in Reference Example B1 were seeded for suspension culture at the time of passage. BioBlu 0.3c Single-Use Vessel (Eppendorf SE) was used as a culture vessel. DASbox (Eppendorf SE) was used as a reactor system for controlling culture. A pH sensor and a medium perfusion pump provided with DASbox were calibrated in advance according to the method specified by the manufacturer. The cells were seeded such that the amount of culture solution was 142 mL and the cell density at the start of culture was 3.0×10⁵ cells/mL, and then the culture was started. StemFit (registered trademark) AK02N (AJINOMOTO CO., INC.) supplemented with 10 μM Y-27632 and 20 μM IWR-1 endo was used as a medium. During the culture, the culture temperature was maintained at 37° C., the carbon dioxide gas concentration in the aeration gas was maintained at 5%, the oxygen concentration was maintained at about 20%, and the aeration gas rate was maintained at 3.0 L/h to perform top surface aeration of the culture solution. The stirring speed was set to 130 rpm until day 1 of culture and 120 rpm afterward. The start of culture was set at day 0 of culture, and medium perfusion was started at day 1 of culture. The amount of medium perfused was changed at 1-hour intervals, as shown in Table 12. The amount of medium perfused per unit time at the start of perfusion (reference perfusion rate) was determined to be F₀=7.40 mL/h obtained by multiplying the result of dividing a culture volume of 142 mL with 24 hours, 5.92 mL, by a constant of 1.25 set based on a cell density of 80% at the start of perfusion with respect to an assumed seeding concentration of 3.0×10⁵ cells/mL. Until day 3 of culture, the amount of medium perfused per unit time was set using Formula 2 setting C₀ as the cell count calculated from a cell density of 80% at the start of perfusion with respect to an assumed seeding concentration of 3.0×10⁵ cells/mL. C was the number of cells calculated from the empirically predicted transition of the assumed cell density. A constant M for correcting the influence of cell lines and the like was set to 1. In addition, after day 3 of culture, Formula 3 was used to set the amount of medium perfused per unit time by calculating K with the amount of lactic acid produced by metabolism per cell per unit time which is assumed from the information in advance as L₀, and the amount of lactic acid produced by metabolism per cell per unit time in each culture time as L on the cell line on day 3 of culture. A constant M for correcting the influence of cell lines and the like was set to 1. The composition of the medium used for perfusion was switched between days 1 to 2 of culture and days 2 to 4 of culture such that the concentration of Y-27632 was different, 4.7 μM for day 1 to day 2 of culture and 2 μM for day 2 to day 4 of culture. StemFit (registered trademark) AK02N (AJINOMOTO CO., INC.) supplemented with 20 μM IWR-1 endo and 1 μM LY333531 was used for both media. To remove the medium and not the cell aggregate by suctioning from the culture solution, the medium was removed by passing through a sintered wire mesh filter having an opening of 30 μm.

TABLE 12

| Elapsed culture time | Flow rate |
|---|---|
| (d. h:m:s) | (mL/h) |
| 1.00:00:00 | 7.40 |
| 1.01:00:00 | 7.65 |
| 1.02:00:00 | 7.91 |
| 1.03:00:00 | 8.18 |
| 1.04:00:00 | 8.46 |
| 1.05:00:00 | 8.74 |
| 1.06:00:00 | 9.04 |
| 1.07:00:00 | 9.34 |
| 1.08:00:00 | 9.66 |
| 1.09:00:00 | 9.99 |
| 1.10:00:00 | 10.33 |
| 1.11:00:00 | 10.68 |
| 1.12:00:00 | 11.04 |
| 1.13:00:00 | 11.41 |
| 1.14:00:00 | 11.80 |
| 1.15:00:00 | 12.20 |
| 1.16:00:00 | 12.61 |
| 1.17:00:00 | 13.04 |
| 1.18:00:00 | 13.48 |
| 1.19:00:00 | 13.94 |
| 1.20:00:00 | 14.41 |
| 1.21:00:00 | 14.90 |
| 1.22:00:00 | 15.41 |
| 1.23:00:00 | 15.93 |
| 2.00:00:00 | 16.47 |
| 2.01:00:00 | 17.03 |
| 2.02:00:00 | 17.60 |
| 2.03:00:00 | 18.20 |
| 2.04:00:00 | 18.82 |
| 2.05:00:00 | 19.46 |
| 2.06:00:00 | 20.12 |
| 2.07:00:00 | 20.80 |
| 2.08:00:00 | 21.50 |
| 2.09:00:00 | 22.23 |
| 2.10:00:00 | 22.98 |
| 2.11:00:00 | 23.76 |
| 2.12:00:00 | 24.57 |
| 2.13:00:00 | 25.40 |
| 2.14:00:00 | 26.26 |
| 2.15:00:00 | 27.15 |
| 2.16:00:00 | 28.07 |
| 2.17:00:00 | 29.02 |
| 2.18:00:00 | 30.01 |
| 2.19:00:00 | 31.03 |
| 2.20:00:00 | 32.08 |
| 2.21:00:00 | 33.16 |
| 2.22:00:00 | 34.29 |
| 2.23:00:00 | 35.45 |
| 3.00:00:00 | 36.65 |
| 3.01:00:00 | 37.64 |
| 3.02:00:00 | 38.66 |
| 3.03:00:00 | 39.70 |
| 3.04:00:00 | 40.76 |
| 3.05:00:00 | 41.85 |
| 3.06:00:00 | 42.97 |
| 3.07:00:00 | 44.12 |
| 3.08:00:00 | 45.30 |
| 3.09:00:00 | 46.50 |
| 3.10:00:00 | 47.73 |
| 3.11:00:00 | 49.00 |
| 3.12:00:00 | 50.29 |
| 3.13:00:00 | 51.62 |
| 3.14:00:00 | 52.98 |
| 3.15:00:00 | 54.37 |
| 3.16:00:00 | 55.80 |
| 3.17:00:00 | 57.26 |
| 3.18:00:00 | 58.75 |
| 3.19:00:00 | 60.28 |
| 3.20:00:00 | 61.85 |
| 3.21:00:00 | 63.45 |
| 3.22:00:00 | 65.09 |
| 3.23:00:00 | 66.77 |

### Comparative Example B1: Suspension Culture of Human iPS Cell Line 1383D6 in Batch Mode

Cell culture was performed under the same conditions as in Comparative Example B1, except that medium change was perform by the batch mode. The culture medium was changed in its entirety every day. StemFit (registered trademark) AK02N (AJINOMOTO CO., INC.) was used as a medium. Table 13 lists the concentrations of additives in the medium used in each medium alter/alteration.

TABLE 13

| | Y-27632 | IWR-1 endo | LY333531 |
|---|---|---|---|
| | (μM) | (μM) | (μM) |
| Day 0 to day 1 of culture | 10 | 20 | 0 |
| Day 1 to day 2 of culture | 6.9 | 20 | 0.58 |
| Day 2 to day 3 of culture | 2.7 | 20 | 0.99 |
| Day 3 to day 4 of culture | 2 | 20 | 1 |

### Comparative Example B2: Suspension Culture of Human iPS Cell Line 1383D6 in Constant Flow Rate Perfusion Mode

Cell culture was performed under the same conditions as in Example B1, except for the amount of medium perfused. The medium perfusion was started on day 1 of culture as in Example B1. The culture was performed at a constant amount of medium perfused of 5.92 mL/h such that the amount of medium used per day was equal to that in Comparative Example B1.

### Evaluation Example B1: Confirmation of Cell Density on Day 4 of Culture

The cell aggregates on day 4 of culture in Example B1 and Comparative Examples B1 and B2 were treated with Accutase (Innovative Cell Technologies, Inc.) for 10 minutes, and unicellularized by pipetting. The cells were suspended in StemFit (registered trademark) AK02N medium containing Y-27632 at a final concentration of 10 μM, and some of the cells were stained with trypan blue to measure the viable cell count. Table 14 and FIG. 9 show the results.

TABLE 14

| | Viable cell density on day |
|---|---|
| | 4 of culture (cells/mL) |
| Example B1 | 2.00 × 10⁶ |
| Comparative Example B1 | 1.26 × 10⁶ |
| Comparative Example B2 | 0.99 × 10⁶ |

As shown in Table 14 and FIG. 9, the cell density of Example B1 is the highest, indicating that the productivity is improved in the perfusion culture method, in which the amount of medium perfused is optimally controlled by the method of the present invention.

### Evaluation Example B2: Confirmation of Specific Growth Rate

The cell aggregates on day 3 of culture in Example B1 and Comparative Examples B1 and B2 were treated with Accutase (Innovative Cell Technologies, Inc.) for 10 minutes, and unicellularized by pipetting. The cells were suspended in StemFit (registered trademark) AK02N medium containing Y-27632 at a final concentration of 10 μM, and some of the cells were stained with trypan blue to measure the viable cell count. Table 15 and FIG. 10 show the results of calculating the specific growth rate for day 3 to day 4 of culture from the measurement results and the results of Evaluation Example B2.

TABLE 15

| | Specific growth rate for day |
|---|---|
| | 3 to day 4 of culture (day⁻¹) |
| Comparative Example B1 | 0.12 |
| Comparative Example B2 | −0.097 |
| Example B1 | 0.67 |

As shown in Table 15 and FIG. 10, the specific growth rate of Example B1 is the highest, indicating that it becomes possible to produce a large amount of cells with high productivity by the perfusion culture method, in which the amount of medium perfused is optimally controlled by the method of the present invention. In addition, it is understood that the amount of production by one-batch can be increased by extending the culture time.

### Evaluation Example B3: Quantitative Real-Time PCR Analysis

Quantitative real-time PCR analysis was performed by the following procedure. The cells on day 4 of culture in Example B1 and Comparative Examples B1 and B2 were dissolved using TRIzol™ Reagent (Thermo Fisher Scientific Inc.). Total RNA was isolated and purified using PureLink (registered trademark) RNA Mini Kit (Thermo Fisher Scientific Inc.) from the solution of the cells dissolved with TRIzol™ Reagent. The concentration of purified RNA was measured using BioSpec-nano (SHIMADZU CORPORATION), thereby fractionating 500 ng of purified RNA. 10 μL solution is prepared with the adding 2 μL of ReverTra Ace (registered trademark) qPCR RT Master mix (TOYOBO CO., LTD.) and Rnase Free dH₂O to the fractionated RNA. cDNA synthesis was performed using SimpliAmp Thermal Cycler (Thermo Fisher Scientific Inc.). Reaction conditions for cDNA synthesis was as follows: a reaction at 37° C. was performed for 15 minutes, and then a reaction at 50° C. for 5 minutes and a reaction at 98° C. for 5 minutes were performed sequentially, followed by cooling to 4° C. The synthesized cDNA solution was diluted 100-fold with 10 mM Tris-HCl (pH 8.0; NACALAI TESQUE, INC.) and added at 5 μL/well to a 384-well PCR plate (Thermo Fisher Scientific Inc.). KOD SYBR (registered trademark) qPCR Mix (TOYOBO CO., LTD.), a forward primer prepared to be 50 μM, a reverse primer prepared to be 50 μM, and DEPC-treated water (NACALAI TESQUE, INC.) were mixed at a ratio of 100:1:1:48. This mixed solution was added at 15 μL/well to the 384-well PCR plate and mixed. ACTB, OCT4, SOX2, NANOG, and HK2 were used as primers. The 384-well PCR plate was centrifuged to remove air bubbles in the wells, and quantitative real-time PCR analysis was performed using QuantStudio 7 Flex Real-Time PCR System (Thermo Fisher Scientific Inc.). Table 16 lists the reaction conditions.

TABLE 16

| Step | Temperature | Time | | Number of cycles |
|---|---|---|---|---|
| 1 Initial denaturation | 98° C. | 1 | min | — |
| 2 Denaturation | 98° C. | 15 | sec | 5 cycle |
| 3 Annealing, Elongation | 68° C. | 30 | sec | |
| 4 Denaturation | 98° C. | 15 | sec | 40 cycle |
| 5 Annealing | 60° C. | 10 | sec | |
| 6 Elongation | 68° C. | 30 | sec | |
| 7 Melting curve | 95° C. | 15 | sec | — |
| | 60° C. | 1 | min | |
| | 98° C. | 15 | sec | |

The base sequences of the primers used for quantitative real-time PCR analysis are shown below.

ACTB (Forward):
(SEQ ID NO: 11)
5′-CCTCATGAAGATCCTCACCGA-3′
ACTB (Reverse):
(SEQ ID NO: 12)
5′-TTGCCAATGGTGATGACCTGG-3′
OCT4 (Forward):
(SEQ ID NO: 13)
5′-AGTGGGTGGAGGAAGCTGACAAC-3′
OCT4 (Reverse):
(SEQ ID NO: 14)
5′-TCGTTGTGCATAGTCGCTGCTTGA-3′
SOX2 (Forward):
(SEQ ID NO: 15)
5′-CACCAATCCCATCCACACTCAC-3′
SOX2 (Reverse):
(SEQ ID NO: 16)
5′-GCAAAGCTCCTACCGTACCAC-3′
NANOG (Forward):
(SEQ ID NO: 17)
5′-AGCCTCCAGCAGATGCAAGAACTC-3′
NANOG (Reverse):
(SEQ ID NO: 18)
5′-TTGCTCCACATTGGAAGGTTCCCA-3′
HK2 (Forward):
(SEQ ID NO: 19)
5′-ACCCAGTTACACACTTACCATC-3′
HK2 (Reverse):
(SEQ ID NO: 20)
5′-TGCAGTGAGCTATGATCATACC-3′

Table 17 and FIG. 11 show the results of gene expression measurement.

TABLE 17
Relative gene expression level to ACTB (2^{-ΔCt})

| | Comparative | Comparative | |
|---|---|---|---|
| | Example B1 | Example B2 | Example B1 |
| OCT4 | 5.83 × 10⁻¹ | 5.15 × 10⁻¹ | 1.02 |
| NANOG | 3.37 × 10⁻² | 3.99 × 10⁻² | 5.67 × 10⁻² |
| SOX2 | 2.26 × 10⁻² | 1.41 × 10⁻² | 3.34 × 10⁻² |
| HK2 | 2.00 × 10⁻³ | 2.82 × 10⁻³ | 4.74 × 10⁻³ |

As shown in Table 17 and FIG. 11, the cells obtained in Example B1 and Comparative Examples B1 and B2 were found to have maintained the undifferentiated property in pluripotent stem cells. The expression levels of the undifferentiation markers and the HK2 gene involved in metabolism tended to be increased in Example B1.

### Evaluation Example B4: Flow Cytometry Analysis

The cell aggregates on day 4 of culture in Example B1 and Comparative Examples B1 and B2 were treated with Accutase and dispersed as single cells by pipetting. The cells were washed with PBS (phosphate-buffered saline). Thereafter, fixation, permeabilization, and blocking were performed using eBioscience Foxp3 Transcription Factor Staining Buffer Set (Thermo Fisher Scientific Inc.). Then, the cell sample was divided into four, and each of them was resuspended to 50 μL using a buffer provided with the eBioscience Foxp3 Transcription Factor Staining Buffer Set (Thermo Fisher Scientific Inc.). Fluorescently-labeled anti-OCT4, anti-SOX2, and anti-NANOG antibodies were added to and mixed with one of the samples, and FMO controls were prepared by mixing each of three samples with antibodies in which each antibody was removed from the three fluorescently-labeled antibodies. Staining was performed at 4° C. for 1 hour in a light-proof state. Table 18 lists the antibodies used and their amounts added.

TABLE 18

| | Manufacturer, Model number | Amount added | |
|---|---|---|---|
| Fluorescently-labeled | Becton, Dickinson and | 10 | μL |
| anti-OCT4 antibody | Company, 560186 | | |
| Fluorescently-labeled | BioLegend, 656110 | 1 | μL |
| anti-SOX2 antibody | | | |
| Fluorescently-labeled | Becton, Dickinson and | 2.5 | μL |
| anti-NANOG antibody | Company, 561300 | | |

After washing once with 3% FBS (fetal bovine serum)/PBS, the cells passed through a cell strainer were analyzed using Guava easyCyte 8HT (Luminex Corporation). For the FMO control samples, all regions where the cell population with the strong fluorescence intensity was 1.0% or less were selected in the cell populations extracted by the FSC/SSC dot plot. For samples treated with the anti-OCT4, anti-SOX2, and anti-NANOG antibodies, the proportion of cells contained within the regions was calculated in the cell populations extracted from the FSC/SSC dot plot. This was defined as the proportion of cells positive for OCT4, SOX2, and NANOG. Table 19 and FIG. 12 show the results.

TABLE 19

| | Proportion of | Proportion of | Proportion of |
|---|---|---|---|
| | cells positive | cells positive | cells positive |
| | for OCT4 (%) | for SOX2 (%) | for NANOG (%) |
| Comparative | 97.1 | 98.5 | 94.6 |
| Example B1 | | | |
| Comparative | 86.4 | 87.3 | 91.1 |
| Example B2 | | | |
| Example B1 | 98.2 | 99.4 | 96.9 |

As shown in Table 19 and FIG. 12, for undifferentiation markers OCT4, SOX2, and NANOG, the cells obtained in Example B1 tend to have a higher positive rate than the cells obtained in Comparative Examples B1 and B2, confirming that not only productivity and also product quality can be improved by the perfusion culture method in which amount of medium perfused is optimally controlled according to the method of the present invention.

### Example B2: Suspension Culture of Human iPS Cell Line 1383D6 in Appropriate Perfusion Mode

The culture was performed in the same manner as in Example B1, except for the cell seeding concentration and the setting of medium perfusion. The cells were seeded such that the cell density at the start of culture was 2.0×10⁵ cells/mL. Table 20 shows the transition of medium perfusion. The medium perfusion was started on day 1 of culture with F₀=5.92 mL/h obtained by multiplying the result of dividing a culture volume of 142 mL with 24 hours, 5.92 mL, by a constant of 1.0 set based on a cell density of 80% at the start of perfusion with respect to an assumed seeding concentration of 2.0×10⁵ cells/mL. Until day 2 of culture, perfusion was performed at a constant volume without applying the method of the present invention. The amount of medium perfused per unit time was set using Formula 2 above setting C₀ as the cell count calculated from a cell density C₀ of 80% at the start of perfusion with respect to an assumed seeding concentration of 2.0×10⁵ cells/mL and an assumed growth rate for day 1 to day 2 of culture according to the method of the present invention from day 2 to day 4.25 of culture. C was the number of cells calculated from the empirically predicted transition of the assumed cell density. A constant M for correcting the influence of cell lines and the like was set to 1. After day 4.25 of culture, the culture was performed at an amount of medium perfused of 33.71 mL/h without applying the method of the present invention in the same manner as for day 1 to day 2 of culture. The composition of the medium used for perfusion was always the same, with a Y-27632 concentration of 2 μM. StemFit (registered trademark) AK02N (AJINOMOTO CO., INC.) supplemented with 20 μM IWR-1 endo was used for both media.

TABLE 20

| Elapsed culture time | Flow rate |
|---|---|
| (d. h:m:s) | (mL/h) |
| 1.00:00:00 | 5.92 |
| 2.00:00:00 | 5.92 |
| 2.01:00:00 | 6.16 |
| 2.02:00:00 | 6.41 |
| 2.03:00:00 | 6.68 |
| 2.04:00:00 | 6.95 |
| 2.05:00:00 | 7.23 |
| 2.06:00:00 | 7.53 |
| 2.07:00:00 | 7.84 |
| 2.08:00:00 | 8.16 |
| 2.09:00:00 | 8.50 |
| 2.10:00:00 | 8.84 |
| 2.11:00:00 | 9.21 |
| 2.12:00:00 | 9.58 |
| 2.13:00:00 | 9.98 |
| 2.14:00:00 | 10.39 |
| 2.15:00:00 | 10.81 |
| 2.16:00:00 | 11.26 |
| 2.17:00:00 | 11.72 |
| 2.18:00:00 | 12.20 |
| 2.19:00:00 | 12.70 |
| 2.20:00:00 | 13.22 |
| 2.21:00:00 | 13.76 |
| 2.22:00:00 | 14.33 |
| 2.23:00:00 | 14.92 |
| 3.00:00:00 | 15.53 |
| 3.01:00:00 | 16.16 |
| 3.02:00:00 | 16.83 |
| 3.03:00:00 | 17.52 |
| 3.04:00:00 | 18.24 |
| 3.05:00:00 | 18.98 |
| 3.06:00:00 | 19.76 |
| 3.07:00:00 | 20.57 |
| 3.08:00:00 | 21.42 |
| 3.09:00:00 | 22.30 |
| 3.10:00:00 | 23.21 |
| 3.11:00:00 | 24.16 |
| 3.12:00:00 | 25.15 |
| 3.13:00:00 | 26.18 |
| 3.14:00:00 | 27.26 |
| 3.15:00:00 | 28.38 |
| 3.16:00:00 | 29.54 |
| 3.17:00:00 | 30.75 |
| 3.18:00:00 | 32.01 |
| 3.19:00:00 | 33.33 |
| 3.20:00:00 | 34.69 |
| 3.21:00:00 | 36.12 |
| 3.22:00:00 | 37.60 |
| 3.23:00:00 | 39.14 |
| 4.00:00:00 | 40.75 |
| 4.01:00:00 | 42.42 |
| 4.02:00:00 | 44.16 |
| 4.03:00:00 | 45.97 |
| 4.04:00:00 | 47.85 |
| 4.05:00:00 | 49.82 |
| 4.06:00:00 | 33.71 |

### Comparative Example B3: Suspension Culture of Human iPS Cell Line 1383D6 in Batch Mode

Cell culture was performed under the same conditions as in Example B2, except that medium change was performed by the batch process. The culture medium was changed in its entirety every day. StemFit (registered trademark) AK02N (AJINOMOTO CO., INC.) was used as a medium. Table 21 lists the concentrations of additives in the medium used in each medium change.

TABLE 21

| | Y-27632 | IWR-1 endo |
|---|---|---|
| | (μM) | (μM) |
| Day 0 to day 1 of culture | 10 | 20 |
| Day 1 to day 2 of culture | 6.9 | 20 |
| Day 2 to day 3 of culture | 2.7 | 20 |
| Day 3 to day 4 of culture | 2 | 20 |
| Day 4 to day 5 of culture | 2 | 20 |
| Day 5 to day 6 of culture | 2 | 20 |

### Comparative Example B4: Suspension Culture of Human iPS Cell Line 1383D6 in Constant Flow Rate Perfusion Mode

Cell culture was performed under the same conditions as in Example B2, except for the amount of medium perfused. The medium perfusion was started on day 1 of culture as in Example B2. The culture was performed at a constant amount of medium perfused of 5.92 mL/h such that the amount of medium used per day was equal to that in Comparative Example B1.

### Evaluation Example B5: Confirmation of Cell Density on Day 6 of Culture

The cell aggregates on day 6 of culture in Example B2 and Comparative Examples B3 and B4 were treated with Accutase (Innovative Cell Technologies, Inc.) for 10 minutes, and unicellularized by pipetting. The cells were suspended in StemFit (registered trademark) AK02N containing Y-27632 at a final concentration of 10 μM, and some of the cells were stained with trypan blue to measure the viable cell count. Table 22 and FIG. 13 show the results.

TABLE 22

| | Viable cell density on day |
|---|---|
| | 6 of culture (cells/mL) |
| Example B2 | 2.18 × 10⁶ |
| Comparative Example B3 | 0.93 × 10⁶ |
| Comparative Example B4 | 0.78 × 10⁶ |

As shown in Table 22 and FIG. 13, the cell density of Example B2 is the highest, indicating that the productivity is improved even in the perfusion culture method, to part of which the method of the present invention is applied.

### Embodiment C: Production of Cell Population Involving Controlling Carbon Dioxide Gas Supply Rate

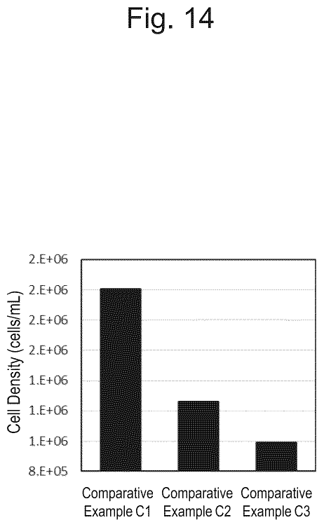

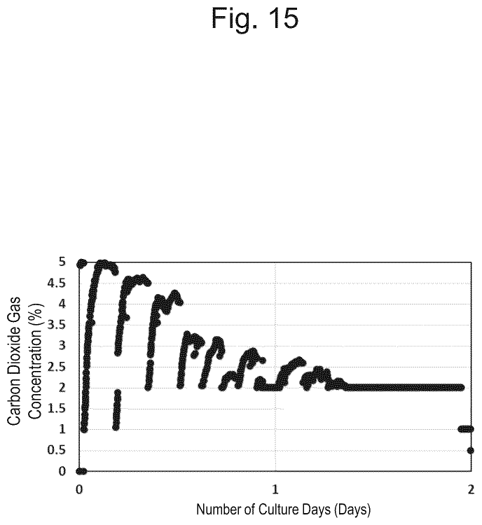

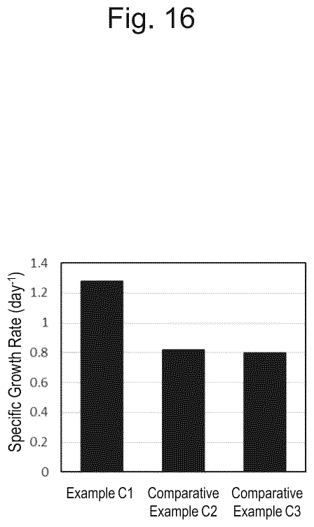

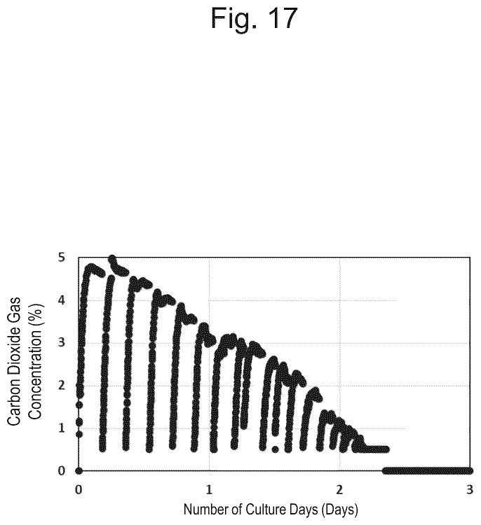

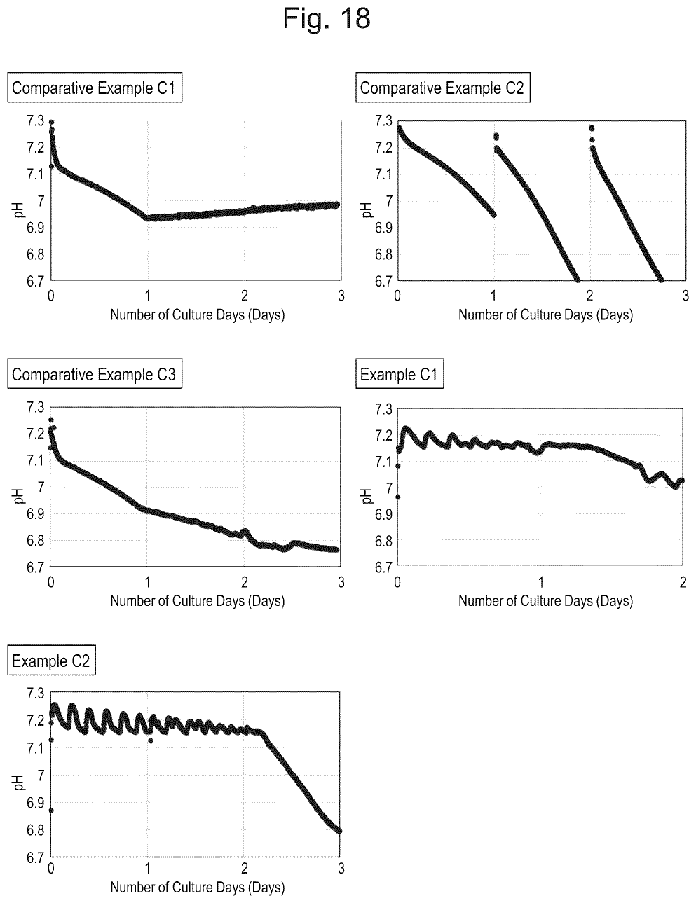

The present embodiment corresponds to the Reference Examples, Comparative Examples, Examples, and Evaluation Examples in Japanese Patent Application No. 2021-051005, on which the priority of the present application is based. To distinguish the Examples and the like from those in the other embodiments, the Examples and the like in the present embodiment are expressed as the Examples and the like in Japanese Patent Application No. 2021-051005 plus “C.” Each of Figures of the present application and Figures in Japanese Patent Application No. 2021-051005 correspond to as follows: FIG. 14 and FIG. 1, FIG. 15 and FIG. 2, FIG. 16 and FIG. 3, FIG. 17 and FIG. 4, and FIG. 18 and FIG. 5. In addition, each of Tables of the present application and Tables in Japanese Patent Application No. 2021-051005 correspond to as follows: Table 23 and Table 1, Table 24 and Table 2, Table 25 and Table 3, Table 26 and Table 4, Table 27 and Table 5, Table 28 and Table 6, Table 29 and Table 7, Table 30 and Table 8, and Table 31 and Table 9. Further, each of SEQ ID NOs of the present application and SEQ ID NOs in Japanese Patent Application No. 2021-051005 correspond to as follows: SEQ ID NO 21 and SEQ ID NO 1, SEQ ID NO 22 and SEQ ID NO 2, SEQ ID NO 23 and SEQ ID NO 3, SEQ ID NO 24 and SEQ ID NO 4, SEQ ID NO 25 and SEQ ID NO 5, SEQ ID NO 26 and SEQ ID NO 6, SEQ ID NO 27 and SEQ ID NO 7, and SEQ ID NO 28 and SEQ ID NO 8.

### Reference Example C1: Adherent Culture of Human iPS Cell Line 1383D6

A human iPS cell line 1383D6 (Center for iPS Cell Research and Application, Kyoto University) was seeded at 10000 cells/cm² on a cell culture dish coated with Vitronectin (VTN-N) Recombinant Human Protein, Truncated (Thermo Fisher Scientific Inc.) at 0.5 μg/cm², followed by adherent culture at 37° C. in a 5% CO₂ atmosphere. StemFit (registered trademark) AK02N (AJINOMOTO CO., INC.) was used as a medium, and medium change was performed every day. Y-27632 (FUJIFILM Wako Pure Chemical Corporation) was added to the medium at a final concentration of 10 μM only at the time of cell seeding. The day of cell seeding was set as day 0 of culture, and cells were detached from the culture surface by treatment with Accutase (Innovative Cell Technologies, Inc.) for 5 minutes and dispersed as single cells by pipetting on day 4 of culture for passage. The cells were suspended in StemFit (registered trademark) AK02N (AJINOMOTO CO., INC.) containing Y-27632 at a final concentration of 10 μM, and some of the cells were stained with trypan blue to measure the viable cell count. Then, cell seeding was performed to continue adherent culture in the same manner.

In the present embodiment, to examine the influence of the presence or absence of control of the amount of carbon dioxide gas supplied on cultured cells, the Example corresponding to Examples A1 and B1 in the previous embodiment is regarded as the Comparative Example (Comparative Example C1) for convenience.

### Comparative Example C1: Suspension Culture of Human iPS Cell Line 1383D6 in Appropriate Perfusion Mode

The cells cultured in Reference Example C1 were seeded for suspension culture at the time of passage. BioBlu 0.3c Single-Use Vessel (Eppendorf SE) was used as a culture vessel. DASbox (Eppendorf SE) was used as a reactor system for controlling culture. A pH sensor and a medium perfusion pump provided with DASbox were calibrated in advance according to the method specified by the manufacturer. The cells were seeded such that the amount of culture solution was 142 mL and the cell density at the start of culture was 3.0×10⁵ cells/mL, and then the culture was started. StemFit (registered trademark) AK02N (AJINOMOTO CO., INC.) supplemented with 10 μM Y-27632 and 20 μM IWR-1 endo was used as a medium. During the culture, the culture temperature was maintained at 37° C., the carbon dioxide gas concentration in the gas supplied was maintained at 5%, the oxygen concentration was maintained at about 20%, and the rate of gas supplied was maintained at 3.0 L/h to perform top surface aeration of the culture solution. The stirring speed was set to 130 rpm until day 1 of culture and 120 rpm afterward. The start of culture was set at day 0 of culture, and medium perfusion was started at day 1 of culture. The amount of medium perfused was changed at 1-hour intervals, as shown in Table 23. The amount of medium perfused per unit time at the start of perfusion (reference perfusion rate) was determined to be F₀=7.40 mL/h obtained by multiplying the result of dividing a culture volume of 142 mL with 24 hours, 5.92 mL, by a constant of 1.25 set based on a cell density of 80% at the start of perfusion with respect to an assumed seeding concentration of 3.0×10⁵ cells/mL. Until day 3 of culture, the amount of medium perfused per unit time was set using Formula 2 above setting C₀ as the cell count calculated from a cell density of 80% at the start of perfusion with respect to an assumed seeding concentration of 3.0×10⁵ cells/mL. C was the number of cells calculated from the empirically predicted transition of the assumed cell density. A constant M for correcting the influence of cell lines and the like was set to 1. In addition, after day 3 of culture, Formula 3 above was used to set the amount of medium perfused per unit time by calculating K with the amount of lactic acid produced by metabolism per cell per unit time which is assumed from the information in advance as L₀, and the amount of lactic acid produced by metabolism per cell per unit time in each culture time as L on the cell line on day 3 of culture. A constant M for correcting the influence of cell lines and the like was set to 1. The composition of the medium used for perfusion was switched between days 1 to 2 of culture and days 2 to 4 of culture such that the concentration of Y-27632 was different, 4.7 μM for day 1 to day 2 of culture and 2 μM for day 2 to day 4 of culture. StemFit (registered trademark) AK02N (AJINOMOTO CO., INC.) supplemented with 20 μM IWR-1 endo and 1 μM LY333531 was used for both media. To remove the medium and not the cell aggregate by suctioning from the culture solution, the medium was removed by passing through a sintered wire mesh filter having an opening of 30 μm.

TABLE 23

| Elapsed culture time | Flow rate |
|---|---|
| (d. h:m:s) | (mL/h) |
| 1.00:00:00 | 7.40 |
| 1.01:00:00 | 7.65 |
| 1.02:00:00 | 7.91 |
| 1.03:00:00 | 8.18 |
| 1.04:00:00 | 8.46 |
| 1.05:00:00 | 8.74 |
| 1.06:00:00 | 9.04 |
| 1.07:00:00 | 9.34 |
| 1.08:00:00 | 9.66 |
| 1.09:00:00 | 9.99 |
| 1.10:00:00 | 10.33 |
| 1.11:00:00 | 10.68 |
| 1.12:00:00 | 11.04 |
| 1.13:00:00 | 11.41 |
| 1.14:00:00 | 11.80 |
| 1.15:00:00 | 12.20 |
| 1.16:00:00 | 12.61 |
| 1.17:00:00 | 13.04 |
| 1.18:00:00 | 13.48 |
| 1.19:00:00 | 13.94 |
| 1.20:00:00 | 14.41 |
| 1.21:00:00 | 14.90 |
| 1.22:00:00 | 15.41 |
| 1.23:00:00 | 15.93 |
| 2.00:00:00 | 16.47 |
| 2.01:00:00 | 17.03 |
| 2.02:00:00 | 17.60 |
| 2.03:00:00 | 18.20 |
| 2.04:00:00 | 18.82 |
| 2.05:00:00 | 19.46 |
| 2.06:00:00 | 20.12 |
| 2.07:00:00 | 20.80 |
| 2.08:00:00 | 21.50 |
| 2.09:00:00 | 22.23 |
| 2.10:00:00 | 22.98 |
| 2.11:00:00 | 23.76 |
| 2.12:00:00 | 24.57 |
| 2.13:00:00 | 25.40 |
| 2.14:00:00 | 26.26 |
| 2.15:00:00 | 27.15 |
| 2.16:00:00 | 28.07 |
| 2.17:00:00 | 29.02 |
| 2.18:00:00 | 30.01 |
| 2.19:00:00 | 31.03 |
| 2.20:00:00 | 32.08 |
| 2.21:00:00 | 33.16 |
| 2.22:00:00 | 34.29 |
| 2.23:00:00 | 35.45 |
| 3.00:00:00 | 36.65 |
| 3.01:00:00 | 37.64 |
| 3.02:00:00 | 38.66 |
| 3.03:00:00 | 39.70 |
| 3.04:00:00 | 40.76 |
| 3.05:00:00 | 41.85 |
| 3.06:00:00 | 42.97 |
| 3.07:00:00 | 44.12 |
| 3.08:00:00 | 45.30 |
| 3.09:00:00 | 46.50 |
| 3.10:00:00 | 47.73 |
| 3.11:00:00 | 49.00 |
| 3.12:00:00 | 50.29 |
| 3.13:00:00 | 51.62 |
| 3.14:00:00 | 52.98 |
| 3.15:00:00 | 54.37 |
| 3.16:00:00 | 55.80 |
| 3.17:00:00 | 57.26 |
| 3.18:00:00 | 58.75 |
| 3.19:00:00 | 60.28 |
| 3.20:00:00 | 61.85 |
| 3.21:00:00 | 63.45 |
| 3.22:00:00 | 65.09 |
| 3.23:00:00 | 66.77 |

### Comparative Example C2: Suspension Culture of Human iPS Cell Line 1383D6 in Batch Mode

Cell culture was performed under the same conditions as in Comparative Example C1, except that medium change was perform by the batch mode. The culture medium was changed in its entirety every day. StemFit (registered trademark) AK02N (AJINOMOTO CO., INC.) was used as a medium. Table 24 lists the concentrations of additives in the medium used in each medium change.

TABLE 24

| | Y-27632 | IWR-1 endo | LY333531 |
|---|---|---|---|
| | (μM) | (μM) | (μM) |
| Day 0 to day 1 of culture | 10 | 20 | 0 |
| Day 1 to day 2 of culture | 6.9 | 20 | 0.58 |
| Day 2 to day 3 of culture | 2.7 | 20 | 0.99 |
| Day 3 to day 4 of culture | 2 | 20 | 1 |

### Comparative Example C3: Suspension Culture of Human iPS Cell Line 1383D6 in Constant Flow Rate Perfusion Mode

Cell culture was performed under the same conditions as in Comparative Example C1, except for the amount of medium perfused. The medium perfusion was started on day 1 of culture as in Comparative Example C1. The culture was performed at a constant amount of medium perfused of 5.92 mL/h such that the amount of medium used per day was equal to that in Comparative Example C1.

### Evaluation Example C1: Confirmation of Cell Density on Day 4 of Culture

The cell aggregates on day 4 of culture in Comparative Examples C1, C2, and C3 were treated with Accutase (Innovative Cell Technologies, Inc.) for 10 minutes, and unicellularized by pipetting. The cells were suspended in StemFit (registered trademark) AK02N (AJINOMOTO CO., INC.) containing Y-27632 at a final concentration of 10 μM, and some of the cells were stained with trypan blue to measure the viable cell count. Table 25 and FIG. 14 show the results.

TABLE 25

| | Viable cell density on day |
|---|---|
| | 4 of culture (cells/mL) |
| Comparative Example C1 | 2.00 × 10⁶ |
| Comparative Example C2 | 1.26 × 10⁶ |
| Comparative Example C3 | 0.99 × 10⁶ |

As shown in Table 25 and FIG. 14, the cell density of Comparative Example C1 is the highest, indicating that the productivity is improved by the appropriate medium perfusion method.

### Example C1: Suspension Culture of Human iPS Cell Line 1383D6 in Appropriate Perfusion Mode with Controlled Carbon Dioxide Gas Concentration

The cells cultured in Reference Example C1 were seeded for suspension culture at the time of passage. BioBlu 0.3c Single-Use Vessel (Eppendorf SE) was used as a culture vessel. DASbox (Eppendorf SE) was used as a reactor system for controlling culture. A pH sensor and a medium perfusion pump provided with DASbox were calibrated in advance according to the method specified by the manufacturer. The cells were seeded such that the culture solution amount was 142 mL and the cell density at the start of culture was 3.0×10⁵ cells/mL, and then the culture was started. StemFit (registered trademark) AK02N (AJINOMOTO CO., INC.) supplemented with 10 μM Y-27632 and 20 μM IWR-1 endo was used as a medium. During the culture, the culture temperature was maintained at 37° C., and the amount of gas supplied was maintained at 3.0 L/h to perform top surface aeration of the culture solution. The carbon dioxide gas concentration in the gas supplied was 5% at the start of culture and then gradually adjusted to decrease to a minimum of 2% as shown in FIG. 15 such that the decrease in the pH of the culture solution is suppressed. The gas supplied was prepared by mixing air with an arbitrary volume of carbon dioxide gas. The stirring speed was set to 130 rpm until day 1 of culture and 120 rpm afterward. The start of culture was set at day 0 of culture, and medium perfusion was started at day 1 of culture. The amount of medium perfused was changed at 1-hour intervals, as shown in Table 26. The amount of medium perfused per unit time at the start of perfusion (reference perfusion rate) was determined to be F₀=6.51 mL/h obtained by multiplying the result of dividing a culture volume of 142 mL with 24 hours, 5.92 mL, by a constant of 1.1 set based on a cell density of 80% at the start of perfusion with respect to an assumed seeding concentration of 3.0×10⁵ cells/mL under culture conditions of this medium and the like. The amount of medium perfused per unit time was set using Formula 3 above as the cell count calculated from a cell density C₀ of 80% at the start of perfusion with respect to an assumed seeding concentration of 3.0×10⁵ cells/mL. C was the number of cells calculated from the empirically predicted transition of the assumed cell density. A constant M for correcting the influence of cell lines and the like was set to 1. In addition, the amount of medium perfused per unit time was set with K calculated by the amount of lactic acid produced by metabolism per cell per unit time which is assumed from the information in advance as L₀, and the amount of lactic acid produced by metabolism per cell per unit time in each culture time as L on the cell line at the start of culture and. A constant M for correcting the influence of cell lines and the like was set to 1. The composition of the medium used for perfusion was switched between days 1 to 2 of culture and days 2 to 4 of culture such that the concentration of Y-27632 was different, 4.7 μM for day 1 to day 2 of culture and 2 μM for day 2 to day 4 of culture. StemFit (registered trademark) AK02N (AJINOMOTO CO., INC.) supplemented with 20 μM IWR-1 endo and 1 μM LY333531 was used for both media. To remove the medium and not the cell aggregate by suctioning from the culture solution, the medium was removed by passing through a sintered wire mesh filter having an opening of 30 μm.

TABLE 26

| Elapsed culture time | Flow rate |
|---|---|
| (d. h:m:s) | (mL/h) |
| 1.00:00:00 | 6.51 |
| 1.01:00:00 | 6.73 |
| 1.02:00:00 | 6.91 |
| 1.03:00:00 | 7.20 |
| 1.04:00:00 | 7.44 |
| 1.05:00:00 | 7.69 |
| 1.06:00:00 | 7.95 |
| 1.07:00:00 | 8.22 |
| 1.08:00:00 | 8.50 |
| 1.09:00:00 | 8.79 |
| 1.10:00:00 | 9.09 |
| 1.11:00:00 | 9.40 |
| 1.12:00:00 | 9.71 |
| 1.13:00:00 | 10.04 |
| 1.14:00:00 | 10.38 |
| 1.15:00:00 | 10.74 |
| 1.16:00:00 | 11.10 |
| 1.17:00:00 | 11.48 |
| 1.18:00:00 | 11.87 |
| 1.19:00:00 | 12.27 |
| 1.20:00:00 | 12.68 |
| 1.21:00:00 | 13.11 |
| 1.22:00:00 | 13.56 |
| 1.23:00:00 | 14.02 |
| 2.00:00:00 | 14.49 |
| 2.01:00:00 | 14.98 |
| 2.02:00:00 | 15.49 |
| 2.03:00:00 | 16.02 |
| 2.04:00:00 | 16.56 |
| 2.05:00:00 | 17.12 |
| 2.06:00:00 | 17.70 |
| 2.07:00:00 | 18.30 |
| 2.08:00:00 | 18.92 |
| 2.09:00:00 | 19.56 |
| 2.10:00:00 | 20.23 |
| 2.11:00:00 | 20.91 |
| 2.12:00:00 | 21.62 |
| 2.13:00:00 | 22.35 |
| 2.14:00:00 | 23.11 |
| 2.15:00:00 | 23.89 |
| 2.16:00:00 | 24.70 |
| 2.17:00:00 | 25.54 |
| 2.18:00:00 | 26.41 |
| 2.19:00:00 | 27.30 |
| 2.20:00:00 | 28.23 |
| 2.21:00:00 | 29.18 |
| 2.22:00:00 | 30.17 |
| 2.23:00:00 | 31.20 |
| 3.00:00:00 | 32.25 |
| 3.01:00:00 | 33.35 |
| 3.02:00:00 | 34.48 |
| 3.03:00:00 | 35.65 |
| 3.04:00:00 | 36.85 |
| 3.05:00:00 | 38.10 |
| 3.06:00:00 | 39.40 |
| 3.07:00:00 | 40.73 |
| 3.08:00:00 | 42.11 |
| 3.09:00:00 | 43.54 |
| 3.10:00:00 | 45.01 |
| 3.11:00:00 | 46.54 |
| 3.12:00:00 | 48.12 |
| 3.13:00:00 | 49.75 |
| 3.14:00:00 | 51.43 |
| 3.15:00:00 | 53.18 |
| 3.16:00:00 | 54.98 |
| 3.17:00:00 | 56.84 |
| 3.18:00:00 | 58.77 |
| 3.19:00:00 | 60.76 |
| 3.20:00:00 | 62.82 |
| 3.21:00:00 | 64.95 |
| 3.22:00:00 | 67.15 |
| 3.23:00:00 | 69.43 |

### Evaluation Example C2: Specific Cell Growth Rate for Day 1 to Day 2 of Culture

The cell aggregates on day 1 and day 2 of culture in Example C1 and Comparative Examples C1 and C3 were treated with Accutase (Innovative Cell Technologies, Inc.) for 10 minutes, and unicellularized by pipetting. The cells were suspended in StemFit (registered trademark) AK02N (AJINOMOTO CO., INC.) containing Y-27632 at a final concentration of 1. μM, and some of the cells were stained with trypan blue to measure the viable cell count. Table 27 and FIG. 16 show the results of calculating the specific growth rate from the measurement results.

TABLE 27

| | Specific growth rate for day |
|---|---|
| | 1 to day 2 of culture (day⁻¹) |
| Example C1 | 1.28 |
| Comparative Example C1 | 0.82 |
| Comparative Example C3 | 0.80 |

As shown in Table 27 and FIG. 16, it is understood that when culture was performed by adjusting the carbon dioxide gas concentration, in addition to the perfusion mode with an appropriate amount of medium perfused, the specific growth rate for day 1 to day 2 of culture was remarkably improved. In cell culture, in general, the initial stage of culture with a low cell density tends to be rate-limiting, and productivity is often inhibited, indicating that the above-described problems can be solved by the present invention.

### Evaluation Example C3: Quantitative Real-Time PCR Analysis

Quantitative real-time PCR analysis was performed by the following procedure. The cells on day 2 of culture in Comparative Examples C1, C2, and C3 and Example C1 were dissolved using TRIzol™ Reagent (Thermo Fisher Scientific Inc.). Total RNA was isolated and purified using PureLink (registered trademark) RNA Mini Kit (Thermo Fisher Scientific Inc.) from the solution of the cells dissolved with TRIzol™ Reagent. The concentration of purified RNA was measured using BioSpec-nano (SHIMADZU CORPORATION), thereby fractionating 500 ng of purified RNA. 10 μL solution is prepared with the adding 2 μL of ReverTra Ace (registered trademark) qPCR RT Master mix (TOYOBO CO., LTD.) and Rnase Free dH₂O to the fractionated RNA. cDNA synthesis was performed using SimpliAmp Thermal Cycler (Thermo Fisher Scientific Inc.). Reaction conditions for cDNA synthesis was as follows: a reaction at 37° C. was performed for 15 minutes, and then a reaction at 50° C. for 5 minutes and a reaction at 98° C. for 5 minutes were performed sequentially, followed by cooling to 4° C. The synthesized cDNA solution was diluted 100-fold with 10 mM Tris-HCl (pH 8.0; NACALAI TESQUE, INC.) and added at 5 μL/well to a 384-well PCR plate (Thermo Fisher Scientific Inc.). KOD SYBR (registered trademark) qPCR Mix (TOYOBO CO., LTD.), a forward primer prepared to be 50 μM, a reverse primer prepared to be 50 μM, and DEPC-treated water (NACALAI TESQUE, INC.) were mixed at a ratio of 100:1:1:48. This mixed solution was added at 15 μL/well to the 384-well PCR plate and mixed. ACTB, OCT4, SOX2, NANOG, and HK2 were used as primers. The 384-well PCR plate was centrifuged to remove air bubbles in the wells, and quantitative real-time PCR analysis was performed using QuantStudio 7 Flex Real-Time PCR System (Thermo Fisher Scientific Inc.). Table 28 lists the reaction conditions.

TABLE 28

| Step | Temperature | Time | | Number of cycles |
|---|---|---|---|---|
| 1 Initial denaturation | 98° C. | 1 | min | — |
| 2 Denaturation | 98° C. | 15 | sec | 5 cycles |
| 3 Annealing, Elongation | 68° C. | 30 | sec | |
| 4 Denaturation | 98° C. | 15 | sec | 40 cycles |
| 5 Annealing | 60° C. | 10 | sec | |
| 6 Elongation | 68° C. | 30 | sec | |
| 7 Melting curve | 95° C. | 15 | sec | — |
| | 60° C. | 1 | min | |
| | 98° C. | 15 | sec | |

The base sequences of the primers used for quantitative real-time PCR analysis are shown below.

ACTB (Forward):
(SEQ ID NO: 21)
5′-CCTCATGAAGATCCTCACCGA-3′
ACTB (Reverse):
(SEQ ID NO: 22)
5′-TTGCCAATGGTGATGACCTGG-3′
OCT4 (Forward):
(SEQ ID NO: 23)
5′-AGTGGGTGGAGGAAGCTGACAAC-3′
OCT4 (Reverse):
(SEQ ID NO: 24)
5′-TCGTTGTGCATAGTCGCTGCTTGA-3′
SOX2 (Forward):
(SEQ ID NO: 25)
5′-CACCAATCCCATCCACACTCAC-3′
SOX2 (Reverse):
(SEQ ID NO: 26)
5′-GCAAAGCTCCTACCGTACCAC-3′
NANOG (Forward):
(SEQ ID NO: 27)
5′-AGCCTCCAGCAGATGCAAGAACTC-3′
NANOG (Reverse):
(SEQ ID NO: 28)
5′-TTGCTCCACATTGGAAGGTTCCCA-3′

Table 29 shows the results of gene expression measurement.

TABLE 29
Relative gene expression level to ACTB (2^{-ΔCt})

| | OCT4 | NANOG | SOX2 |
|---|---|---|---|
| Example C1 | 5.21 × 10⁻¹ | 1.19 × 10⁻² | 3.00 × 10⁻² |

As shown in FIG. 29, the expression level of each gene showing the undifferentiated property of the cells obtained in Example C1 was sufficient, indicating that pluripotent stem cells maintained the undifferentiated property.

### Example C2: Suspension Culture by Constant Flow Rate Perfusion Mode Combined with Adjustment of Carbon Dioxide Gas Concentration of Human iPS Cell Line 1383D6

The culture was performed under the same conditions as in Comparative Example C3, except that the carbon dioxide gas concentration was adjusted in line with the progress of culture, and cells were seeded such that the cell density at the start of culture was 2.0×10⁵ cells/mL. The carbon dioxide gas concentration at the start of culture was 5% and then gradually adjusted to decrease to a minimum of 0% as shown in FIG. 17 such that the decrease in the pH of the culture solution is suppressed.

### Evaluation Example C4: Cell Survival Rate on Day 3 of Culture

The cell aggregate on day 3 of culture, and thus after the carbon dioxide gas concentration in Example C2 reached 0%, was treated with Accutase (Innovative Cell Technologies, Inc.) for 10 minutes, and unicellularized by pipetting. The cells were suspended in StemFit (registered trademark) AK02N (AJINOMOTO CO., INC.) containing Y-27632 at a final concentration of 10 μM, and some of the cells were stained with trypan blue to measure the viable cell rate. Table 30 shows the results.

TABLE 30

| | Cell viability in culture |
|---|---|
| | medium on day 3 of culture (%) |
| Example C2 | 94 |

As shown in Table 30, the cell survival rate in the culture solution after the carbon dioxide gas concentration reached 0% exhibits a high value equivalent to the value in general cell culture.

### Evaluation Example C5: Quantitative Real-Time PCR Analysis

Gene expression levels of OCT4, NANOG, and SOX2 in cells on day 3 of culture, and thus after the carbon dioxide gas concentration in Example C2 reached 0%, were measured in the same manner as in Evaluation Example C3. Table 31 shows the results.

TABLE 31
Relative gene expression level to ACTB (2^{-ΔCt})

| | OCT4 | NANOG | SOX2 |
|---|---|---|---|
| Example C2 | 6.32 × 10⁻¹ | 1.47 × 10⁻² | 4.29 × 10⁻² |

As shown in Table 31, it was confirmed that the expression level of each gene in the cells in the culture solution after the carbon dioxide gas concentration reached 0% was sufficiently high, indicating that the undifferentiated property was maintained. In general, it is common sense to supply a certain volume of carbon dioxide gas during cell culture, and choosing not to supply carbon dioxide gas is discouraged due to the risk of adverse effects on cells. However, as shown in Evaluation Examples C4 and C5, even when the carbon dioxide gas concentration was gradually decreased until the supply was finally stopped, the cells survived without dying, and the undifferentiated property maintained.

### Evaluation Example C6: Measurement of Culture Environment Parameter

As an example of a parameter representing the culture environment during the culture in Comparative Examples C1, C2, and C3 and Examples C1 and C2, pH values were measured using a pH sensor provided with the reactor system DASbox. FIG. 18 shows the results.

As shown in FIG. 18, it was confirmed using pH as an example of parameter that it is possible to appropriately maintain the culture environment at a constant level by combining the perfusion culture method at a suitable amount of medium perfused with the culture method involving adjusting the concentration of carbon dioxide gas supplied.

All publications, patents, and patent applications cited herein are incorporated herein by reference in their entirety.

1. A method for producing a pluripotent stem cell population, comprising:
a suspension culture step of performing a suspension culture of pluripotent stem cells in a liquid medium in a perfusion mode; and
a passage step of passaging the pluripotent stem cells subjected to the suspension culture,
wherein the suspension culture step comprises controlling the amount of medium perfused per unit time in a range of 1% to 100% of a culture volume.

2. The method according to claim 1, wherein the amount of medium perfused per unit time is determined based on a value obtained by multiplying the culture volume by a proportion of a length of the unit time to 24 hours.

3. The method according to claim 1, wherein the control in the suspension culture step is based on one or more culture variables.

4. The method according to claim 3, wherein the control allows the amount of medium perfused per unit time to be proportional to each of the one or more culture variables.

5. The method according to claim 3, wherein one of the culture variables is a cell density increasing rate, the control includes increasing the amount of medium perfused per unit time based on an increase in the cell density increasing rate, and the cell density increasing rate represents a proportion of a cell density to a cell density of the pluripotent stem cells at the start of the control.

6. The method according to claim 3, wherein one of the culture variables is a pH of a culture solution in which the pluripotent stem cells are present, and the control includes changing the amount of medium perfused per unit time to suppress a decrease in the pH.

7. The method according to claim 6, wherein the decrease in the pH is suppressed by increasing the amount of medium perfused per unit time based on the decrease in the pH.

8. (canceled)

9. The method according to claim 3, wherein one of the culture variables is a lactic acid production rate of the pluripotent stem cells, and the lactic acid production rate is maintained in a range of 1.0×10⁻¹⁰ mmol/cell/h to 2.5×10⁻⁹ mmol/cell/h.

10. The method according to claim 1, wherein a lactic acid concentration in the culture solution in which the pluripotent stem cells are present is 10 mM or less at the start of the control.

11. The method according to claim 1, wherein the pluripotent stem cell population comprises a cell aggregate, and the cell aggregate is collected during the suspension culture step.

12. The method according to claim 1, wherein
the pluripotent stem cell population comprises a cell aggregate, and
in the suspension culture step, perfusion is started by the perfusion mode after the cell aggregate is formed.

13. The method according to claim 1, wherein
the pluripotent stem cell population comprises a cell aggregate, and
one of the culture variables is a cell aggregate volume increasing rate, and the cell aggregate volume increasing rate represents a proportion of a cell aggregate volume to a cell aggregate volume at the start of the control.

14. The method according to claim 1, wherein the control includes increasing an amount of medium perfused for any 6 hours of culture after the cell density of the pluripotent stem cells reached 8.0×10⁵ cells/mL compared to an amount of medium perfused for 6 hours of culture immediately therebefore.

15. (canceled)

16. The method according to claim 1, wherein the suspension culture step comprises altering an amount of carbon dioxide gas supplied to the liquid medium based on one or more indicators.

17. The method according to claim 16, wherein the alteration includes changing a carbon dioxide gas concentration in a gas supplied to the liquid medium within a range of 0% to 10% carbon dioxide gas concentration.

18. The method according to claim 16, wherein one of the indicators is a pH of the culture solution in which the pluripotent stem cells are present, and the alteration includes changing the carbon dioxide gas concentration in a gas supplied to the liquid medium so as to suppress a decrease in the pH.

19. (canceled)

20. The method according to claim 13, wherein the pluripotent stem cell population comprises a cell aggregate, and the carbon dioxide gas concentration in a gas supplied to the liquid medium is started to be changed before the cell aggregate is formed.

21. The method according to claim 1, wherein a specific growth rate of cells before the passage step relative to the growth rate after the passage step is 0.2 to 1.5 day⁻¹.

22-29. (canceled)

30. The method according to claim 1, wherein a proportion of cells positive for OCT4 is 90% or more, a proportion of cells positive for SOX2 is 90% or more, and a proportion of cells positive for NANOG is 90% or more in the pluripotent stem cell population.

31. The method according to claim 1, wherein the pluripotent stem cells are ES cells and/or induced pluripotent stem cells.


SEQUENCE LISTING (28 sequences)

SEQ ID NO: 1 - DNA, 21 bp, Artificial
  Synthetic DNA
cctcatgaagatcctcaccga

SEQ ID NO: 2 - DNA, 21 bp, Artificial
  Synthetic DNA
ttgccaatggtgatgacctgg

SEQ ID NO: 3 - DNA, 23 bp, Artificial
  Synthetic DNA
agtgggtggaggaagctgacaac

SEQ ID NO: 4 - DNA, 24 bp, Artificial
  Synthetic DNA
tcgttgtgcatagtcgctgcttga

SEQ ID NO: 5 - DNA, 22 bp, Artificial
  Synthetic DNA
caccaatcccatccacactcac

SEQ ID NO: 6 - DNA, 21 bp, Artificial
  Synthetic DNA
gcaaagctcctaccgtaccac

SEQ ID NO: 7 - DNA, 24 bp, Artificial
  Synthetic DNA
agcctccagcagatgcaagaactc

SEQ ID NO: 8 - DNA, 24 bp, Artificial
  Synthetic DNA
ttgctccacattggaaggttccca

SEQ ID NO: 9 - DNA, 22 bp, Artificial
  Synthetic DNA
acccagttacacacttaccatc

SEQ ID NO: 10 - DNA, 22 bp, Artificial
  Synthetic DNA
tgcagtgagctatgatcatacc

SEQ ID NO: 11 - DNA, 21 bp, Artificial
  Synthetic DNA
cctcatgaagatcctcaccga

SEQ ID NO: 12 - DNA, 21 bp, Artificial
  Synthetic DNA
ttgccaatggtgatgacctgg

SEQ ID NO: 13 - DNA, 23 bp, Artificial
  Synthetic DNA
agtgggtggaggaagctgacaac

SEQ ID NO: 14 - DNA, 24 bp, Artificial
  Synthetic DNA
tcgttgtgcatagtcgctgcttga

SEQ ID NO: 15 - DNA, 22 bp, Artificial
  Synthetic DNA
caccaatcccatccacactcac

SEQ ID NO: 16 - DNA, 21 bp, Artificial
  Synthetic DNA
gcaaagctcctaccgtaccac

SEQ ID NO: 17 - DNA, 24 bp, Artificial
  Synthetic DNA
agcctccagcagatgcaagaactc

SEQ ID NO: 18 - DNA, 24 bp, Artificial
  Synthetic DNA
ttgctccacattggaaggttccca

SEQ ID NO: 19 - DNA, 22 bp, Artificial
  Synthetic DNA
acccagttacacacttaccatc

SEQ ID NO: 20 - DNA, 22 bp, Artificial
  Synthetic DNA
tgcagtgagctatgatcatacc

SEQ ID NO: 21 - DNA, 21 bp, Artificial
  Synthetic DNA
cctcatgaagatcctcaccga

SEQ ID NO: 22 - DNA, 21 bp, Artificial
  Synthetic DNA
ttgccaatggtgatgacctgg

SEQ ID NO: 23 - DNA, 23 bp, Artificial
  Synthetic DNA
agtgggtggaggaagctgacaac

SEQ ID NO: 24 - DNA, 24 bp, Artificial
  Synthetic DNA
tcgttgtgcatagtcgctgcttga

SEQ ID NO: 25 - DNA, 22 bp, Artificial
  Synthetic DNA
caccaatcccatccacactcac

SEQ ID NO: 26 - DNA, 21 bp, Artificial
  Synthetic DNA
gcaaagctcctaccgtaccac

SEQ ID NO: 27 - DNA, 24 bp, Artificial
  Synthetic DNA
agcctccagcagatgcaagaactc

SEQ ID NO: 28 - DNA, 24 bp, Artificial
  Synthetic DNA
ttgctccacattggaaggttccca